(19) United States
(12) Patent Application Publication
(10) Pub. No.: US 2018/0169253 A1
(43) Pub. Date: Jun. 21, 2018

(54) Means and Methods for Treatment of B-Cell Malignancies

(71) Applicant: UNIVERSITAET DES SAARLANDES, Saarbruecken (DE)

(72) Inventor: Michael Pfreundschuh, Homburg (DE)

(21) Appl. No.: 15/579,312

(22) PCT Filed: Jun. 10, 2016
(86) PCT No.: PCT/EP2016/063246

§ 371 Date: Dec. 4, 2017

(30) Foreign Application Priority Data
Jun. 10, 2015 (EP) 15171498.7
Jun. 10, 2015 (LU) 92737

(51) Int. Cl. A61K 47/64; A61P 35/00; G01N 33/574; C07K 16/28

(52) U.S. Cl. CPC A61K 47/64; A61P 35/00; C07K 2317/622; C07K 16/2809; G01N 33/574

(57) ABSTRACT

The present invention provides means and methods for diagnosing or treating B-cell malignancies. Novel conjugates for reverse targeting of B-cell receptors on malignant B cells are provided. The conjugates comprise a BCR antigen and a diagnostic or therapeutic agent. Also provided herein is a method for identifying patients disposed to respond favorably to treatment with a BCR antigen conjugate of the invention.

### BACKGROUND

B-cell malignancies—malignant diseases of the blood-forming organs, marked by distorted proliferation and development of B-cells and their precursors in the lymph nodes, blood and/or bone marrow—represent a heterogeneous group of disorders with widely varying characteristics and clinical behavior that still account for more than 15,000 deaths annually in the United States.

Each B-cell malignancy is the consequence of genetic and epigenetic anomalies occurring at different stages of B-cell lymphopoiesis. B-cell lymphomas, malignant neoplasms of lymphiod tissue characterized by malignant transformation of B lymphocytes or their precursors, include both Hodgkin Lymphoma and non-Hodgkin Lymphomas (NHL), B-cell leukemias, a group of malignant neoplasms of hematopoietic tissues are characterized by aberrant proliferation and development of B-cells and their precursors in the blood and bone marrow, are among the most common childhood cancers. The dynamic expression of various surface markers during B cell development provides the necessary growth, differentiation, maturation and survival signals. When a B cell at any given stage becomes malignant, the expression pattern of its cell surface molecules and associated intracellular signaling molecules, and the over-expressed gene products associated with this developmental stage is passed on to its clonal malignant derivatives.

Despite advances in clinical care, many mature B-cell malignancies remain incurable. Historically, most affected patients received a combination of cytotoxic agents often in combination with radiation therapy with the intent of achieving a durable remission and, in some cases, a cure. However, many traditional regimens are also associated with considerable acute and long-term toxicities. Stem cell transplantation, often the last alternative for patients suffering from acute forms or developing chemotherapy resistances, comes with a considerable risk of life-threatening rejection reactions. Recent therapeutic approaches focus on monoclonal antibodies, such as rituximab (anti-CD20), antibody-conjugates and CAR T cells recognizing B-cell surface antigens. Despite improvements in survival for many, long-term remission is rare and disease relapse is still a major problem. In addition, most surface antigens targeted by conventional biological therapeutics are not exclusively expressed on the malignant B-cells accounting for the disease, and therefore entail the risk of massive—and potentially life-threatening—side effects due to targeting other (healthy) cells expressing the same surface molecules. In addition, many targeted surface markers are not expressed on malignant B-cells in sufficiently large numbers and thus do not allow their effective depletion. Moreover, most binding molecules such as antibodies are rather large, which negatively affects their biodistribution and bioavailability, and their production is relatively costly.

In sum, effective treatment is thus still hampered by the lack of specificity of the available therapeutic options, which often result in severe side effects and fail to effectively eliminate all malignant B-cells in the patient.

WO 2010/085345 A1 further generically discloses human phosphorylated paratarg or epitopes thereof coupled to therapeutic, cytotoxic or diagnostic agents. Paratarg (stomatin-like protein 2) has been identified as a common antigenic target recognized by secreted paraprotein (i.e. abnormal immunoglobulin fragments produced in excess by an abnormal clonal proliferation of terminally differentiated plasma cells), in patients suffering from monoclonal gammopathy of undetermined significance (MGUS), a condition bound by the presence of markedly increased levels of paraprotein in the blood. However; considering the excess of paraprotein in the blood of said patients, acting as non-functional paratarg “scavengers”, the approach is highly unlikely to be feasible in patients for two reasons: 1st, the malignant cell in MGUS is a plasma cell and plasma cells do not carry a B-cell receptor on their surface. Thus, the BCR could not target the malignant cell in this disease but would rather be bound and trapped by secreted “decoy” paraproteins without even reaching the malignant plasma cells producing the paraproteins. 2nd, both MGUS and MM are characterized by the fact that high amounts of soluble BCR in the form of immunoglobulins, i. e. the paraproteins, are secreted into and present in the serum of the respective patients. Systemic application of recombinant BCR antigen (i.e. paratarg) would result in the formation of immune complexes with a high risk of immune complex disease. Using toxin-conjugated BCR antigens would result in the deposition of toxic immune complexes in the endothelial cells of various organs causing considerable damage and presumable necrosis of these organs, e. g. immune complex nephritis, immune complex vasculitis and other immune complex-mediated inflammatory reactions.” Therefore, from WO 2010/085345 A1, it could clearly not been foreseen that an antigen conjugate targeting the surface-expressed BCR instead of non-functional secreted paraprotein would be useful for treating B-cell malignancies as described herein, i.e. characterized by malignant B cells expressing a functional surface BCR and preferably not associated with gammopathy (excess amount of paraprotein in the blood).

In view of the above, there is an urgent need in the art to provide novel improved therapeutics for treating B cell malignancies that are specific, safe and effective.

### SUMMARY

The invention relates to a B-cell receptor (BCR) antigen conjugated to a diagnostic and/or therapeutic agent that is intended for use in a method of diagnosis and/or treatment of a B-cell malignancy in a patient. It is envisaged that the B-cell malignancy is characterized by the presence of malignant B-cells, in particular clonal malignant B-cells, that express a BCR, in particular a functional BCR expressed on the cell surface.

The conjugated BCR antigen can be used for treatment of a variety of B-cell malignanies, including Chronic lymphocytic leukemia(CLL), B-cell prolymphocytic leukemia, Mantle cell lymphoma (MCL), diffuse large B cell lymphoma (DLBCL), splenic marginal zone lymphoma (SMZL), Burkitt's lymphoma, B-cell non-Hodgkin's lymphoma, follicular lymphoma, primary central nervous system lymphoma (PCNSL), or nodular lymphocyte predominant Hodgkin's lymphoma (NLPHL), hairy cell leukemia, splenic lymphoma, lymphoplasmacytic lymphoma, extranodal marginal zone lymphoma of mucosa-associated lymphoid tissue (MALT lymphoma), nodal marginal zone lymphoma, pediatric nodal marginal zone lymphoma, primary cutaneous follicle centre lymphoma, T-cell/histiocyte rich large B-cell lymphoma, lymphomatoid granulomatosis, primary mediastinal (thymic) large B-cell lymphoma, intravascular large B-cell lymphoma, ALK-positive large B-cell lymphoma, plasmablastic lymphoma, or primary effusion lymphoma.

The BCR antigen may comprise a peptide, polypeptide, protein, lipid or polysaccharide. It may be selected from an autoantigen, an exogenous antigen, an alloantigen or a heteroantigen. The BCR may be posttranslationally modified or unmodified.

It is contemplated that the BCR of the malignant B-cells in the patient recognizes the conjugated BCR antigen, and/or that the conjugated BCR antigen is capable of binding to said B-cells, in particular via their BCR. The conjugated BCR antigen may also be internalized by malignant B-cells after binding their BCR. It is further envisaged that the conjugated BCR antigen of the invention is capable of reducing the number of malignant B-cells expressing a BCR in the patient.

The conjugated BCR antigen is envisaged to be able to elicit one or more of the following advantageous effects: reduce the number of malignant B-cells, inhibit activation and/or proliferation of malignant B-cells, and/or kill malignant B-cells.

The therapeutic agent conjugated to the BCR antigen may be selected from a radionuclide, a binding agent, a CAR T cell, or a toxin. Suitable toxins include chemical toxins, chemotherapeutic agents, and protein toxins. The binding agent may in particular be capable of binding to an immunologic effector cell, e.g. a natural killer cell, macrophage, (cytotoxic) T-cell or neutrophil. Particularly suitable binding agents for use in the BCR conjugate of the invention include CD3 binding agents, CD16 binding agents, or CD38 binding agents. The binding agents described herein may be selected from a monoclonal antibody, a polyclonal antibody, a single chain antibody, a ScFv, a minibody, a Fv, a Fab, a Fab′, or a F(ab′)2 fragment.

Chemotherapeutic agents useful as therapeutic agents in the BCR antigen conjugate of the invention include cytostatic agents such as enidyene, duocarmycin, methothrexate, doxorubicin, melphalan, chlorambucil, ARA-C, vindesine, mitomycin C, cisplatin, etoposide, bleomycin, vedotin, emtansin, 5-fluorouracil or tyrosine kinase inhibitors.

Also provided herein is the use of a BCR antigen conjugated to a diagnostic and/or therapeutic agent in method of diagnosis and/or treatment of B-cell malignancies as defined herein.

Further, the present invention relates to a method for diagnosing or treating B-cell malignancies in a patient as defined herein, comprising
(i) providing a BCR antigen conjugated to a diagnostic agent or a therapeutic agent, preferably as defined herein;
(ii) administering said conjugated BCR antigen to the patient

The invention also provides a BCR antigen conjugated to a therapeutic agent, preferably as defined herein; which is capable of binding to the BCR of malignant B-cells in a patient suffering from a B-cell malignancy as defined herein, and which may further be capable of becoming internalized after binding of the BCR to the conjugated BCR antigen.

Further, the invention provides a pharmaceutical composition comprising a conjugated BCR antigen as defined herein, and a pharmaceutically acceptable excipient.

In a further aspect, the invention relates to a non-invasive method for identifying a patient suffering from a B-cell malignancy as defined herein, being disposed to respond favorably to a conjugated BCR antigen of the invention.

### DESCRIPTION OF THE FIGURES

FIG. 1: Demonstration of ARS2 binding and internalization by the ABC-DLBCL cell line OCI-Ly3 which carries a BCR with specificity for ARS2. A: Preparation of cells; B-D: flow cytometry at t=0; E-G: flow cytometry after one hour of incubation (t=1).

FIG. 2: Proliferation inhibition of ABC-DLBCL lymphoma cells by toxin-conjugated BARs. A recombinant ARS2/pseudomonas exotoxin conjugate (ARS-ETA) specifically kills cells from ABC-DLBCL cell lines with a BCR specific for ARS2 (U2932, OCI-LY3), while ABC-DLBCL cells with BCR specificities other than for ARS2 (HBL-1, TMD8) are unaffected. A control BAR/toxin conjugate of Neurabin1 (the antigenic target of ⅔ of primary CSN lymphomas), NRB1-ETA exerts no toxicity against any of the cell lines.

FIG. 3: BCR-specific killing of OCY-L3 cells by Pseudomonas-exotoxin-conjugated ARS2 in vitro. Only the ARS2-toxin kills the OCY-L3 that have a BCR specific for ARS2; as a control, the pseudomonas-exotoxin-conjugated neurabin (the antigen specific for BCR from ⅔ of CNS lymphomas) leaves the OCY-L3 cells unaffected.

FIG. 4: Specific cytotoxicity of recombinant pseudomonas-exotoxin conjugated ARS2 against an ABC-DLBCL cell line with BCR specific for ARS2 (OCI-Ly3) in vivo. 5×106 OCI-Ly3 cells were injected into the flanks of SCID beige mice on day 9. On day 6 mice received a single intravenous injection of 15 μg toxin-conjugated ARS2 or toxin-conjugated neurabin. Green curves represent the tumor volume (±SEM) of 3 mice receiving ARS2 toxin, red curves the tumor volume (±SEM) of 3 mice receiving the control toxin (pseudomonas-exotoxin conjugated neurabin, the BCR antigen of ⅔ of primary CNS lymphomas).

FIG. 5: Binding of the bispecific CD3-ARS2 construct to DLBCL Cell Lines. A: U2932 (ARS2 reactive), B: HBL-1 (not ARS2 reactive), C: OCI-Ly3 (ARS2 reactive). Color code: red=lymphoma cells+PBS; black=lymphoma cells+ARS2; green=lymphoma cells+ARS2-CD3. The construct binds specifically and exclusively to cell lines expressing a BCR with specificity for ARS2

FIG. 6: Binding of CD3-ARS2 to peripheral blood monocytes (PBMC). A: Binding to PBMC of a healthy control. The results show that the CD3-ARS construct binds to CD4-positive and CD8-positive T-cells in the peripheral blood of a healthy donor. B: double staining with CD4-FITC and CD3-ARS (APC). C: double staining with CD8-FITC and CD3-ARS (APC).

FIG. 7: CD3-ARS-2 mediated cytotoxicity of peripheral blood monocytes (T-cells) against different diffuse large B-cell lines. The cytotoxic effects are only observed of cell lines expressing a BCR with specificity against ARS-2 (OCILy3, U2932) A-C: Different lymphoma cell lines and PBMCs were incubated with CD3-ARS2 at different concentrations. Cytotoxicity was assessed by determining LDH release according to the manufacturer's instructions.

### DETAILED DESCRIPTION

Herein, the present inventors provide a novel approach of targeting neoplastic B-cells in B-cell malignancies using toxic baits for their B-cell receptor (BCR). Naturally, the main function of the BCR on the surface of normal (and malignant) B-cells is the recognition and binding of its specific antigen. After binding of the antigen, the B-cell receptor/antigen complex is rapidly internalized. The antigen is processed and fragments thereof are presented to T-cells in the context of the MHC-complex on the surface of the B-cell as an antigen-presenting cell.

After binding to the BCR, the BCR antigen stimulates the B-cell and induces proliferation. Chronic antigenic stimulation of the B-cell via its BCR has been proposed as a mechanism involved in the development of B-cell lymphomas, and early in their development B-cell lymphomas depend on the antigen for survival. The present inventors envisaged that since binding of the BCR antigen to the BCR results in rapid internalization (“eating”) of the BCR/antigen-complex, the BCR antigen can be used as a deadly bait when being coupled to an agent that can kill the lymphoma cell. Indeed, the inventors found that toxin-conjugated BCR antigens can inhibit or kill targeted B-cells in vitro, and even in vivo as demonstrated by a marked decrease in the size of tumors of ABC-DLBCL cells in SCID mice. These findings clearly came as a surprise, as it is well known in the art that encounter of a B-cell with its specific antigen results in activation and proliferation of the B-cell. Thus, it could not be foreseen that a conjugated BCR antigen would indeed be capable of acting on the contrary and inhibiting or even killing malignant B-cells. This principle is also novel, because only few BCR antigens have been molecularly defined to date, and exclusively as a result of research into the etiology and pathogenesis of B-cell lymphomas. Using BCR antigens as therapeutic agents for the treatment of B-cell malignancies has not been suggested to date. In a systematic search for BCR antigens, the present inventors pioneered in identifying dominant BCR antigens in a variety of B-cell malignancies. For all these types of B-cell malignancies, BCR antigen conjugates represent a novel therapeutic option, and could satisfy an unmet medical need.

Because BCR are abundant on the surface of a lymphoma cell (roughly ten times more frequent than the CD20 molecule which is the target of the monoclonal antibody rituximab which is part of the standard first line treatment of all B-cell lymphomas), the B-cell lymphoma cells are highly sensitive to specific conjugated BCR antigens. Moreover, because the BCR antigen is specific to the individual lymphoma cells and not found on any other B cells or other cells of the body, such a treatment has high and ultimate specificity. BCR antigen conjugates as provided herein therefore have a unique specificity, because they target only the malignant cells, leaving all other cells of the body unaffected.

This should be of particular importance if the conjugated BCR antigen is used as part of a CAR-T-cell or as a bispecific construct. CAR T-cells and bispecific antibodies like blinatumomab are potent therapeutic strategies, but are very toxic, probably due to unspecific activation by normal cross-reacting cells.

As another advantage, BCR binding epitopes may consist only of a small number of molecules, e.g. a peptide spanning 10 to 20 amino acids, thereby keeping production costs low. Due to the potential small size of the conjugate, the conjugates provide great flexibility and a variety of opportunities with respect to pharmacological modifications (e. g. for the penetration of the blood-brain barrier).

The approach presented herein is also designated “BARs”, an acronym for “B-cell receptor antigens for reverse targeting”, because this approach reverses the widely used antibody strategy, where antibodies bind to antigens on malignant cells, while BARs are antigens that bind to the BCR on the surface of a B-cell lymphoma cell. As the affinity of antibodies can be increased by mutations, it is expected that mutations might also increase the affinity of BARs to their BCR.

With the expected low toxicity and high efficacy of the BCR antigen conjugates, it is assumed that the approach presented herein will eventually become part of the standard first-line treatment, which for most of the B-cell lymphomas consists of R-CHOP (combination chemotherapy and rituximab). Addition of the BCR antigen conjugates to standard R-CHOP should be feasible, but substitution of components of the R-CHOP combination (most likely candidate: vincristine) can also be envisaged in subpopulations of patient with a cure rate close to 100% (e.g. young patients with favorable DLBCL), where less toxicity becomes an issue.

The present invention therefore provides a B-cell receptor (BCR) antigen conjugated to a diagnostic and/or therapeutic agent to be used in a method of diagnosis and/or treatment of a B-cell malignancy in a patient.

### BCR Antigen Conjugate

The “B-cell receptor antigen conjugated to a diagnostic and/or therapeutic agent” is also abbreviated “conjugated BCR antigen”, “BCR antigen conjugate”, “BAR” or “conjugate” herein and denotes an antigen conjugated, i.e. linked, to a therapeutic and/or diagnostic agent and preferably recognized by the BCR of malignant B-cells present in the patient. The conjugate of the invention is also said to comprise an “antigenic part”, or “antigenic target”, i.e. the BCR antigen, and a “therapeutic/diagnostic part”, i.e. the therapeutic or diagnostic agent. The term “antigen” refers to a molecule or a portion of a molecule capable of being bound by a selective binding agent, such as a BCR. An antigen may have one or more epitopes. The term “conjugated” in all its grammatical forms means that the antigen and the agent are linked or bound to each other by one or more of the following: one or more covalent bonds, one or more ionic-bonds, one or more permanent dipole bonds, one or more instantaneous dipole to induced dipole bonds (van der Waals). The terms “coupled (to)”, “linked (to)” “conjugated (to)” are used interchangeably herein. In particular, the BCR antigen conjugate is envisaged to be able to bind to the BCR of the malignant B-cells present in patients suffering from a B-cell malignancy. Depending on the intended mode of action, e.g. when coupled to a toxin, the BCR antigen conjugate should be internalized be the malignant B-cell, and preferably release the toxin in order to kill the malignant B-cell. However, when coupled e.g. to a binding agent, a CAR T cell or a diagnostic agent, it may be sufficient if the BCR antigen conjugate binds to the BCR of the malignant B-cells, thereby bringing the malignant cell and the therapeutic or diagnostic agent into close proximity, so that detection of the malignant cell (e.g. in case a diagnostic agent is conjugated to the BCR antigen) or killing of the malignant cell (e.g. in case a CAR T cell or an antibody capable of binding and activating a cytotoxic effector cell, including, but not limited to T-cells, natural killer cells, macrophages or neutrophils is conjugated to the BCR antigen) is possible.

### B-Cells and BCR

The term “B-cell” or “B lymphocyte” in its broadest sense comprises a pre-B cell, immature B cell, a mature naive B cell, a mature activated B cell, a memory B cell, a B lineage lymphocyte, or any other B lineage cell. The skilled person will readily understand that—as it is the principle underlying the present invention to target a “toxic bait” (i.e., BCR antigen conjugate) to the BCR of malignant B-cells present in the patient—the malignant B-cells preferably express a BCR, preferably a functional BCR on the cell surface that is preferably capable of recognizing, i.e., binding to, the BCR antigen conjugate of the present invention. That is, within the context of the present invention the term “B-cell” is envisaged to preferably exclude plasma cells, i.e. terminally differentiated cells of the B-cell lineage that secrete antibodies at a high rate and known to lack the expression of a (functional) B-cell receptor (BCR) on their surface. The B-cell may further be characterized by the expression of specific cell surface markers, including CD19, CD20, CD22, CD23, CD79, IgM, IgD, IgG or IgA as identifiable by flow cytometry.

The “B-cell receptor” or “BCR” is a transmembrane receptor protein complex exclusively expressed by B cells and comprising clonally variable antigen-binding portions—the heavy and light immunoglobulin chains—associated with, a heterodimer called Ig-α/Ig-β (CD79), bound together by disulfide bridges. Each member of the dimer spans the plasma membrane and has a cytoplasmic tail bearing an immunoreceptor tyrosine-based activation motif (ITAM). The antigen-binding portion of the B-cell receptor complex is a cell-surface immunoglobulin that has the same antigen specificity as the secreted antibodies that the B cell will eventually produce. Thus the complete B-cell receptor is thought to be a complex of six chains—two identical light chains, two identical heavy chains, one Igα, and one Igβ. Without wishing to be bound by theory, it is thought that the BCR is preferably expressed by immature B-cells, naïve B-cells and mature B-cells, whereas B-cell receptor expression on the cell surface may be lost in plasma cells.

### B-Cell Malignancies

The term “B-cell malignancy” or “B-cell neoplasm” in its broadest sense refers to a malignancy or neoplasm of B cells, i.e. derived from any stage of a B cell. The term encompasses B-cell lymphomas, B-cell leukemias, and myelomas. A B-cell malignancy in accordance with the present invention is characterized by the presence of malignant B-cells, preferably clonal malignant B-cells expressing a BCR. “Malignant” cells are generally not self-limited in their growth, are capable of invading into adjacent tissues, and may be capable of spreading to distant tissues (metastasizing). “Malignant” when used herein is synonymous with “cancerous”. When used herein, the term “malignant B-cell” is in particular envisaged to refer to a B-cell that can evade apoptosis, displays self-sufficiency of growth signals, and/or exhibits insensitivity to antigrowth signals, as ascertainable using routine methods known in the art. E.g., evasion of apoptosis can be determined as reviewed in Elmore S Toxicol Pathol. 2007; 35(4): 495-516, for example by evaluating caspase activity. As described elsewhere herein, B-cell malignancies envisioned for treatment with the BCR antigen conjugate of the present invention include particularly those not associated with a clonal malignant plasma cells, and/or gammopathy.

The term “clonal” B-cells refers to a group of B-cells that are preferably descended from and in general genetically identical with a single progenitor. “In general genetically identical” means that clonal B-cells may comprise spontaneous mutations differentiating them from each other. However, it is envisaged that the clonal malignant B-cells express a BCR having the same antigen specificity, i.e. recognizing the same antigen, that can thus be targeted with the same BCR antigen conjugate.

The presence of clonal malignant B-cells can be evaluated using routine methods known in the art. The presence of clonal malignant B-cells is usually first suspected by the appearance of enlarged lymph nodes or by the presence of lymphocytosis, an increase in a type of white blood cell, on a complete blood count (CBC) test. Further, microscopic examination and/or flow cytometry can be used to demonstrate an abnormal (i.e., clonal malignant) population of B lymphocytes in the patient, usually in a sample obtained from the blood, bone marrow, lymph nodes or extralymphatic tissues etc., depending on the type of B-cell malignancy. Malignant B-cells often express an atypical but characteristic pattern of molecules on the cell surface, as ascertainable by flow cytometry using specific antibodies with fluorescent labels recognizing said marker molecules. Clonality can usually be inferred by the detection of only one of the mutually exclusive immunglobulin light chains, kappa or lambda, on the entire population of the abnormal B cells. Normal B lymphocytes consist of a stew of different immunglobulin-producing cells, resulting in a mixture of both kappa and lambda expressing cells. The lack of the normal distribution of kappa and lambda producing B cells is one basis for demonstrating clonality.

The conjugated BCR antigen of the invention is contemplated to be useful for treatment of a variety of B-cell malignancies, preferably those caused by and associated with the presence of clonal malignant B cells expressing a functional BCR on their surface, as explained elsewhere herein. Such malignancies include, without limitation, Chronic lymphocytic leukemia(CLL), B-cell prolymphocytic leukemia, Mantle cell lymphoma (MCL), diffuse large B cell lymphoma (DLBCL), splenic marginal zone lymphoma (SMZL), Burkitt's lymphoma, B-cell non-Hodgkin's lymphoma, follicular lymphoma, primary central nervous system lymphoma (PCNSL), or nodular lymphocyte predominant Hodgkin's lymphoma (NLPHL), hairy cell leukemia, splenic lymphoma, lymphoplasmacytic lymphoma, extranodal marginal zone lymphoma of mucosa-associated lymphoid tissue (MALT lymphoma), nodal marginal zone lymphoma, pediatric nodal marginal zone lymphoma, primary cutaneous follicle centre lymphoma, T-cell/histiocyte rich large B-cell lymphoma, lymphomatoid granulomatosis, primary mediastinal (thymic) large B-cell lymphoma, intravascular large B-cell lymphoma, ALK-positive large B-cell lymphoma, plasmablastic lymphoma, or primary effusion lymphoma.

It will be understood that as the conjugated BCR antigen of the invention is intended to target the BCR of the malignant B-cells present in the patient, treatment with said conjugate will preferably result in inhibition or killing of malignant B-cells, in particular clonal malignant B-cells expressing a BCR, preferably a functional BCR on the cell surface. B-cell malignancies are typically of complex etiology, and some B-cell malignancies (e.g. MGUS) are not only characterized by the presence of malignant B-cells expressing a BCR (i.e., progenitor cells to antibody-producing plasma cells), but also by the presence of malignant B-cells not expressing a BCR (e.g., antibody producing plasma cells). Without wishing to be bound by theory, it is envisioned that in such cases, malignant B-cells expressing a BCR, in particular a functional BCR on the cell surface, can be targeted with the BCR-antigen conjugate of the present invention, while malignant B-cells not expressing a BCR, in particular a functional BCR on the cell surface, cannot. Treatment with the BCR-antigen conjugate of the present invention is therefore thought to reduce the number of B-cells expressing a BCR, in particular a functional BCR on the cell surface, in the patient, while additional treatment may be required to eliminate B-cells not expressing such BCR. If the B-cell malignancy to be treated is e.g. characterized by the presence of (functional surface) BCR expressing malignant B-cells that are progenitor cells to malignant non-(functional surface) BCR expressing B-cells, then it is envisioned that treatment with the BCR antigen conjugate of the invention may be particularly useful for inhibiting or killing of the progenitor cells, thereby preferably eliminating the source of production of malignant daughter B-cells and/or preventing a relapse of the patient. Thus, the conjugates of the invention could in principle also be used for consolidation of MGUS/MM in remission; i.e. when the number of paraprotein producing plasma cells has been reduced significantly and the inventive conjugates are less likely to be bound and trapped by secreted “decoy” paraproteins without reaching the malignant plasma cells producing the paraproteins.

However, it is particularly envisaged herein to treat B cell malignancies that are caused by and associated with expansion of clonal malignant B-cells carrying a functional BCR on their surface, and not of plasma B-cells secreting antibodies or antibody fragments (as is the case in MM/MGUS). That is so because, the conjugated BCR antigen is preferably envisaged to be bound by the surface-expressed BCR on the clonal malignant B cells, instead of being “snatched away” and depleted by binding to secreted antibodies or fragments thereof (such as paraprotein). B-cell malignancies that are particularly envisaged for treatment include diffuse large B-cell lymphoma (DLBL), mantle cell lymphoma (MTL), primary central nervous system lymphoma (PCNSL), chronic lymphocytic leukemia (CLL), follicular lymphoma, as long as the disease is not caused by or associated with clonal malignant plasma cells or their antibody-producing precursors. That is, the aforementioned diseases envisaged for treatment with the BCR antigen conjugate are preferably not associated with gammopathy/excess production of (non-functional) immunoglobulins.

As set out herein, the B-cell malignancies to be treated with the conjugated BCR antigen of the invention are preferably characterized in the presence of clonal malignant B-cells expressing a BCR, particularly a functional BCR on the cell surface. Cell surface expression of the BCR can easily be determined using routine methods in the art, e.g. using flow cytometry with labeled antibodies specific for the constant regions of the surface immunoglobulins. A BCR is termed “functional” herein if it has one or more of the following properties: i) specific recognition of an antigen, and, after recognizing or binding the antigen, ii) internalization of the BCR-BCR antigen conjugate complex. The functional BCR may further exhibit one or more of the following capabilities after binding to its specific antigenic target: iii) phosphorylation of the immunoreceptor tyrosine based activation motifs (ITAMs) in Igα/β iv) phosphorylation of downstream signaling components Syk, Bruton's tyrosine kinase (Btk), and/or phospholipase Cγ2 (PLCγ2) v) production of second messengers DAG and/or IP3 vi) release of Ca²⁺ from intracellular stores.

Internalization of the BCR-BCR antigen conjugate complex can be visualized using fluorescence microscopy and labeled conjugates as described elsewhere herein.

Phosphorylation of Igα/β ITAMs, Syk, Brk or PLCγ2 is readily ascertainable using routine techniques, such as immunoprecipitation and immunoblotting using commercially available antibodies. Kits for detecting intracellular Ca2+ or DAG release are also commercially available.

It is thus envisioned that the conjugated BCR antigen may be internalized by the malignant B-cells after binding to the BCR of the malignant B-cells. Internalization (or endocytosis) of the BCR-BCR antigen conjugate complex can be assessed using routine methods known in the art and as set out in the appended examples. Internalization is thought to be important in particular when the BCR antigen-conjugate comprises a toxin that can exert its function (i.e. inhibiting or killing of the malignant B-cell) when it becomes internalized and preferably released from the BCR antigen conjugate within the cell.

### BCR Antigen

As described herein in detail, the present invention is based on the idea to target malignant B-cells in a patient using a “toxic bait”, i.e. a BCR antigen-conjugate, that will be specifically recognized by said malignant B-cells, eventually resulting in inhibition or death of said cells. In general, any antigen can be used as part of the BCR antigen conjugate of the present invention, as long as it is capable of binding to (i.e., being recognized by) the BCR of the malignant B-cells. In cases where internalization of the BCR-antigen conjugate of the invention is required to exert the advantageous capabilities of the BCR antigen (i.e., inhibiting or killing malignant B-cells), the antigen should preferably allow internalization of said conjugate. The antigenic targets recognized by the BCR of the malignant B-cells in the patient can be found using protein arrays. E.g., B-cells can be obtained from a sample (e.g., from the blood, bone marrow, lymph nodes or extralymphatic tissues of the patient) using standard protocols, and subjected to papain digestion, yielding Fab fragments of the BCRs. Subsequently, the Fab fragments can be screened for antigen binding using a (commercially available) protein array, e.g. Unipex 1 and Unipex 2 (Source Biosciences). Screening of the Fab fragments can be repeated with different protein arrays until an antigenic target is found. Because of the abundancy of malignant B-cells typically present in patients suffering from a B-cell neoplasm, antigenic targets of malignant B-cells can be easily identified by detecting the bound Fab fragments with a (fluorescently) labeled secondary (anti-Fab) antibody. Surprisingly, despite of the immensely large number of antigens that could theoretically be recognized by the BCRs of malignant B-cells found in B-cell malignancies, the present inventors have found that dominant antigens, i.e. antigenic targets shared in a number of patients suffering from the same B-cell malignancy, exist for a variety of B-cell neoplasms.

Exemplary dominant antigens found by the present inventors are set out in table 1:

Notably, as will be appreciated by the skilled person in the art, the BCR-antigen conjugate does not necessarily have to comprise the complete molecule identified as an antigenic target (e.g., protein), but may comprise only a fragment of the identified antigenic target (e.g., peptide), as long as said fragment is capable of binding to the BCR of the malignant B-cells, i.e. preferably comprises one or more epitopes recognized by said BCR.

Peptides, polypeptides, proteins, lipids and polysaccharides are particularly envisaged as antigenic targets, i.e. as antigenic parts of the conjugate of the invention. The term “peptide” refers to a compound consisting of two to ten amino acids linked by chemical bonding between their respective carboxyl and amino groups (i.e., peptide bond), whereas a “polypeptide” consists of more than 10 amino acids and a “protein” consists of more than 100 amino acids. The term “peptide” encompasses native peptides (e.g. degradation products or recombinant peptides) and peptidomimetics (synthetically synthesized peptides), including peptoids and semipeptoids. Similarly, the terms “polypeptide” and “protein” encompass native, synthetically synthesized and recombinant polypeptides or proteins, respectively.

It is also conceivable to use modified BCR antigens that are altered to include, e.g., an improved or additional functionality, e.g. reduced immunogenicity, increased or decreased hydrodynamic size (size in solution), solubility and/or stability and/or extended serum half-life. Exemplary functional moieties for use in accordance with the invention include peptides or protein domains binding to other proteins in the human body (such as serum albumin, the immunoglobulin Fc region or the neonatal Fc receptor (FcRn), polypeptide chains of varying length (e.g., XTEN technology or PASylation®), non-proteinaceous polymers, including, but not limited to, various polyols such as polyethylene glycol (PEGylation), polypropylene glycol, polyoxyalkylenes, or copolymers of polyethylene glycol and polypropylene glycol, or of carbohydrates, such as hydroxyethyl starch (e.g., HESylation®) or polysialic acid (e.g., PolyXen® technology).

The term “lipid” encompasses fats and fat-like substances, including triglycerides, fatty acids, neutral fats, phospholipids, glycolipids, waxes, and steroids. The term “polysaccharide” refers to a carbohydrate polymer that is formed from three or more molecules of monosaccharides. It is envisioned that polysaccharides comprising repeating carbohydrate epitopes are particularly suitable as polysaccharide antigenic targets.

The antigen may be an endogenous antigen, in particular an autoantigen, or an exogenous antigen. “Endogenous antigens” are produced from within the host's cells as part of normal cell metabolism or when the cells are infected by (intracellular) bacteria or viruses. An “autoantigen” (self-antigen) is an endogenous constituent of a host that is capable of evoking an immune response by the same. It is envisioned that said autoantigen, in particular if it is a peptide, polypeptide or protein, may be posttranslationally modified or unmodified. “Post-translational modifications” are modifications that occur on a protein after its translation by ribosomes is complete. “Post-translational modification” includes the (covalent) addition of functional groups to a protein, but also refers to proteolytic processing and folding processes necessary for a protein to mature functionally. Conceivable posttranslational modifications in the context of the present invention include, without limitation, phosphorylation, palmitoylation, glycosylation, ubiquitinylation, SUMOylation, methylation, acetylation, and decarboxylation or the respective reverse processes (de-phosphorylation, de-acetylation, de-mythlation etc.).

The antigen may also be an exogenous antigen, i.e. an antigen that enters the body of the host from the outside, e.g. through inhalation, ingestion, or injection. Exogenous antigens include particles considered foreign within the host. The term also comprises “heterologous antigens” or “heteroantigens”, i.e. antigens originating from a species foreign to the host. Exemplary exogenous antigens comprise allergens (such as pollen), or parts of microorganisms (such as coat, capsule, cell wall, flagella, fimbria, or toxin of bacteria, viruses, etc.).

It is further envisaged that the antigen may be an alloantigen, i.e. a foreign antigen from members of the same species (e.g., humans).

It is further envisaged that the antigen of the BCR antigen conjugate of the invention comprises at least one epitope recognized by the BCR of the clonal malignant B-cells. The term “epitope” includes any determinant, particularly a region of an antigen, capable of specific binding to an immunoglobulin, such as a BCR. Epitope determinants include chemically active surface groupings of molecules such as amino acids, sugar side chains, phosphoryl or sulfonyl, and may have specific three-dimensional structural characteristics, and/or specific charge characteristics. Notably, it is envisioned that the BCR-antigen conjugate may comprise only a fragment of the antigen (i.e., antigenic fragment), which preferably comprises one or more epitopes recognized by the BCR of the malignant B-cells.

Immunological binding generally refers to the non-covalent interactions of the type which occur between an immunoglobulin molecule (as in the BCR) and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. In general, a molecule, such as an immunoglobulin molecule, is said to exhibit “specific binding” if it reacts or associates more frequently, more rapidly, with greater duration and/or with greater affinity with a particular substance than it does with alternative substances. Thus, a BCR is herein said to “specifically” bind to a BCR-antigen conjugate (or, more specifically, the antigen part of the conjugate) when it preferentially recognizes its target antigen in a complex mixture of molecules, i.e. reacts with its antigen with greater affinity, avidity, more readily, and/or with greater duration than it does with other substances. Methods to determine such specific binding are also well known in the art.

The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. A BCR may in particular specifically bind to a BCR antigen conjugate (or, more specifically, the antigenic part of the conjugate) when the equilibrium dissociation constant is ≤10⁻⁷ or less, preferably 10⁻⁸ M or less, or more ≤10⁻⁹ M or less.

The conjugated BCR antigen of the present invention is envisioned to have various advantageous capabilities. It is envisaged to inhibit activation and/or proliferation of malignant B-cells in the patient (also shortly termed “inhibition” or “inhibiting” herein). This can be accomplished, e.g., by bringing into close proximity with a cytostatic agent, or by recruiting other cells that inhibit activation and/or proliferation. Additionally or alternatively, the conjugate of the invention is envisioned to kill malignant B-cells in the patient. Killing can be accomplished by one of several mechanisms, such as by induction of apoptosis, bringing into close proximity a toxin, or attracting other cells such as cytotoxic T lymphocytes or macrophages that can kill the targeted B-cells. Killing of the malignant B-cells is ascertainable using routine methods known in the art, e.g. as set out in the appended examples.

Preferably by inhibiting and/or killing malignant B-cells, the conjugated BCR antigen of the invention is also contemplated to eventually reduce the number of malignant B-cells in the patient.

### Conjugated Agents

As set out herein, the present invention envisages a BCR antigen conjugated to a therapeutic or diagnostic agent. Exemplary therapeutic agents particularly useful for treatment of B-cell malignancies according to the invention include radionuclides, binding agents, CAR T-cells, and toxins.

### Radionuclides

A radionuclide is a nuclide that is radioactive and is also referred to as a radioisotope or radioactive isotope. Suitable radionuclides can, e.g., be selected from alpha-emitting isotopes such as 225Ac, 211At, 212Bi, 213Bi, 212Pb, 224Ra or 223Ra. Alternatively, the cytotoxic radionuclide can a beta-emitting isotope such as 186Rh, 188Rh, 177Lu, 90Y, 131I, 67Cu, 64Cu, 153Sm or 166Ho. Other cytotoxic radionuclides conceivable for use as toxins emit Auger and low energy electrons and can be selected from 125I, 123I or 77Br.

The radionuclide for use in the present invention is preferably is an alpha-emitting isotope such as or ²²⁵Ac, ²¹¹At, ²¹²Bi, ²¹³Bi, ²¹²Pb, ²²⁴Ra or ²²³Ra. Alternatively, the radionuclide may a beta-emitting isotope such as ¹⁸⁶Rh, ¹⁸⁸Rh, ¹⁷⁷Lu, ⁹⁰Y, ¹³¹I, ⁶⁷Cu, ¹⁵³Sm or ¹⁶⁶Ho. Further, the radionuclide may emit Auger and low energy electrons and may be one of the isotopes ¹²⁵I, ¹²³I or ⁷⁷Br.

### Binding Agent

It is also envisioned to couple a “binding agent” to the BCR antigen of the present invention. Binding agents can e.g. be used to recruit immune effector cells to the malignant B-cell. Immune effector cells are preferably non-B cells, e.g. natural killer (NK) cells, macrophages, neutrophils or (cytotoxic) T cells. For example, while the antigenic part of the BCR antigen conjugate targets malignant B-cells, a conjugated CD16 binding agent is thought to be capable of recruiting natural killer (NK) cells to the malignant B-cell, which will eventually kill the B-cell. Other binding agents capable of recruiting NK cells or other immune effector cells capable of killing or inhibiting the malignant B-cell are also envisaged herein, e.g. CD3 binding agents, and CD38 binding agents. Preferred binding agents are isolated polypeptides, in particular isolated antibodies, or antigen-binding fragments thereof. The skilled person will readily understand that the binding agents chosen as part of the BCR antigen conjugate of the present invention preferably do not (e.g., sterically) interfere with binding of the BCR of malignant B-cells to the antigenic part of the conjugate, so as to retain the capability of the BCR antigen conjugate to function as an “adapter” between the malignant cells and the recruited immune effector cells, thereby yielding a BCR-antigen conjugate preferably having the same advantageous properties as described herein (i.e., binding to the BCR of malignant B-cells and killing or inhibiting said cells).

The term “antibody” refers to a monoclonal or a polyclonal antibody which binds to an antigenic target, or a derivative of said antibody which retains or essentially retains its binding specificity. Exemplary derivatives of such antibodies are chimeric antibodies comprising, for example, a mouse or rat variable region and a human constant region, and humanized antibodies, i.e. antibodies of non-human origin, where at least one complementarity determining region (CDR) in the variable regions such as the CDR3 and preferably all 6 CDRs have been replaced by CDRs of an antibody of human origin having a desired specificity, and, optionally, the non-human constant region(s) of the antibody has/have been replaced by (a) constant region(s) of a human antibody.

The term “antibody” also encompasses functional antibody fragments, i.e. fragments of antibodies which retain or essentially retain the binding specificity of the antibodies like, separated light and heavy chains, Fab, Fab/c, Fv, Fab′, F(ab′)2. The term “antibody” also comprises bifunctional (bispecific) antibodies and antibody constructs, like single-chain Fvs (scFv), tandem-scFv, bis-scFvs, or antibody-fusion proteins, heavy chain antibodies and the variable domains thereof, domain antibodies (“dAb's”), which are based on or derived from the heavy chain variable domain (VH) or the light chain variable domain (VL) of traditional 4 chain antibody molecules, diabodies, single-chain antibody molecules; and multispecific antibodies formed from antibody fragments.

Bispecific B-cell antigen conjugates comprising an antigen and a binding agent also termed “Bi-BARs” herein.

### Toxin

The therapeutic agent coupled to the antigen in the conjugate of the invention may be a toxin. The term “toxin” as used herein encompasses, without limitation, chemical toxins, chemotherapeutic agents and protein toxins. Any other compound is also conceivable as long as it can be conjugated to a BCR antigen as described herein, yielding a BCR conjugate preferably having the same advantageous capabilities as described herein (i.e., binding to the BCR of malignant B-cells and killing or inhibiting said cells).

“Chemotherapeutic agents” that are conceivable for use with the present invention comprise any antineoplastic and/or cytostatic agent useful in the treatment of B-cell malignancies, including small sized organic molecules, peptides, oligonucleotides and the like. Agents included in the definition of chemotherapeutic agents are, without limitation, alkylating agents, e.g. mechlorethamine, cyclophosphamide, melphalan, chlorambucil, ifosfamide, busulfan, N-Nitroso-N-methylurea (MNU), carmustine (BCNU), lomustine (CCNU), semustine (MeCCNU), fotemustine, streptozotocin, dacarbazine, mitozolomide, temozolomide, thiotepa, mytomycin, diaziquone (AZQ), cisplatin, carboplatin, oxaliplatin, procarbazine and hexamethylmelamine; antimetabolites, e.g. methotrexate, pemetrexed, fluorouracil, capecitabine, cytarabine, gemcitabine, decitabine, Vidaza, fludarabine, nelarabine, cladribine, clofarabine, pentostatin, thioguanine, mercaptopurine; anti-microtubule agents e.g. vincristine, vinblastine, vinorelbine, vindesine, vinflunine, paclitaxel, docetaxel, podophyllotoxin; topoisomerase inhibitors, e.g. irinotecan, topotecan, etoposide, doxorubicin, mitoxantrone, teniposide, novobiocin, merbarone, aclarubicin; cytotoxic antibiotics, e.g. actinomycin, bleomycin, plicamycin, mitomycin, daunorubicin, epirubicin, idarubicin, pirarubicin, aclarubicin, mitoxantrone; poisonous lectins; plant toxins, e.g. ricin, abrin, modeccin; botulina toxins, diphtheria toxins; enidyene, duocarmycin, calicheamicin, esperamicin, doxorubicin, ARA-C, cis-platinum, 5-fluorouracil, dolastatin, auristatin, vedotin, emtansin; tyrosine kinase inhibitors, or any other small molecule drug known in the art for treatment of the B-cell malignancies as defined herein. Derivatives and combinations thereof are also envisioned for use in the BCR conjugate of the invention. Particularly preferred toxins in accordance with the invention include, without limitation, enidyene, duocarmycin, methotrexate, doxorubicin, melphalan, chlorambucil, ARA-C, vindesine, mitomycin C, cisplatin, etoposide, bleomycin, vedotin, emtansin, 5-fluorouracil and tyrosine kinase inhibitors (e.g. imatinib). CAR T cell

CAR T cells are T-cells carrying artificial chimeric T cell receptors (or chimeric antigen receptors (CARs)). Typically, these receptors are used to graft the specificity of a monoclonal antibody onto a T cell. The most common form of these molecules are fusions of single-chain variable fragments (scFv) derived from monoclonal antibodies, fused to CD3-ζ transmembrane- and endodomains. Upon recognition of the scFv of its antigenic target, such CARs result in the transmission of signals inducing activation, proliferation, production of cytokines and/or effector functions of the CAR-T cell.

CAR T-cells employed as therapeutic agents in the BCR conjugates of the invention are envisaged to be targeted to the malignant B-cells by the antigenic part of the conjugate binding to the BCR, where the CAR T cell will eventually be activated and inhibit or kill the malignant B-cell.

BCR antigen conjugates comprising a CAR-T cell as a therapeutic agent are also termed “BAR-CARs” herein.

### Chemical Modification

The conjugated BCR antigen may be chemically modified. Generally, all kind of modifications are conceivable, as long as they do not abolish the advantageous properties of the conjugated BCR antigen of the invention, i.e. the chemically modified BCR antigen conjugates should preferably have capabilities which are comparable to the capabilities of the compounds which were evaluated in the appended examples. In particular, modified BCR antigen conjugates should preferably retain their ability of binding to the BCR of malignant B-cells, inhibiting or killing said B-cells. The capability of being internalized may further be desired.

### Diagnostic Agent

(Detectable) labels can be attached to the BCR antigen for diagnostic purposes. Such labels conceivable for use in accordance with the invention include, without limitation, fluorescent molecules, or magnetic entities, such as magnetic particles. Thereby, the presence and/or concentration of the target in a sample can be detected by detecting the signal produced by the detectable label.

A detectable label can be detected directly or indirectly, and several different detectable labels conjugated to different specific-antibodies can be used in combination to detect one or more targets.

Examples of detectable labels, which may be detected directly, include fluorescent labels, enzyme labels, radioisotopes, chemiluminescent labels, electrochemiluminescent labels, bioluminescent labels, polymers, polymer particles, metal particles, haptens, and dyes. Examples of fluorescent labels include 5-(and 6)-carboxyfluorescein, 5- or 6-carboxyfluorescei n, 6-(fluorescein)-5-(and 6)-carboxamido hexanoic acid, fluorescein isothiocyanate, rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, green fluorescent protein (GFP) and analogues thereof, and conjugates of R-phycoerythrin or allophycoerythrin, inorganic fluorescent labels such as particles based on semiconductor material like coated CdSe nanocrystallites. Examples of polymer particle labels include micro particles or latex particles of polystyrene, PMMA or silica, which can be embedded with fluorescent dyes, or polymer micelles or capsules which contain dyes, enzymes or substrates. Examples of metal particle labels include gold particles and coated gold particles, which can be converted by silver stains. Examples of haptens include DNP, fluorescein isothiocyanate (FITC), biotin, and digoxigenin. Examples of enzymatic labels include horseradish peroxidase (HRP), alkaline phosphatase (ALP or AP), β-galactosidase (GAL), glucose-6-phosphate dehydrogenase, β-N-acetylglucosamimidase, β-glucuronidase, invertase, Xanthine Oxidase, firefly luciferase and glucose oxidase (GO). Examples of commonly used substrates for horseradishperoxidase include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), .alpha.-naphtol pyronin (.alpha.-NP), o-dianisidine (OD), 5-bromo-4-chloro-3-indolylphosp-hate (BCIP), Nitro blue tetrazolium (NBT), 2-(p-iodophenyl)-3-p-nitropheny-I-5-phenyl tetrazolium chloride (INT), tetranitro blue tetrazolium (TNBT), 5-bromo-4-chloro-3-indoxyl-beta-D-galactoside/ferro-ferricyanide (BCIG/FF). Examples of commonly used substrates for Alkaline Phosphatase include Naphthol-AS-B 1-phosphate/fast red TR (NABP/FR), Naphthol-AS-MX-phosphate/fast red TR (NAMP/FR), Naphthol-AS-B1-phosphate/-fast red TR (NABP/FR), Naphthol-AS-MX-phosphate/fast red TR (NAMP/FR), Naphthol-AS-B1-phosphate/new fuschin (NABP/NF), bromochloroindolyl phosphate/nitroblue tetrazolium (BCIP/NBT), 5-Bromo-4-chloro-3-indolyl-b-d-galactopyranoside (BCIG). Examples of luminescent labels include luminol, isoluminol, acridinium esters, 1,2-dioxetanes and pyridopyridazines. Examples of electrochemiluminescent labels include ruthenium derivatives. Examples of radioactive labels include radioactive isotopes of iodide, cobalt, selenium, tritium, carbon, sulfur and phosphorous.

BCR antigens conjugated to both a therapeutic and a diagnostic agent are also envisaged herein and could e.g. be used for monitoring distribution and internalization of the conjugate in the patient, while at the same time targeting the malignant B-cells.

### Linkers

The therapeutic or diagnostic agent is typcially attached to the BCR antigen via a linker. Conceivable linkers include, for instance In general, linkers include flexible, cleavable and rigid linkers and will be selected depending on the type of conjugate and intended use/application. For example for therapeutic application, non-immunogenic, flexible linkers are often preferred in order to ensure a certain degree of flexibility or interaction between the domains while reducing the risk of adverse immunogenic reactions. Such linkers are generally composed of small, non-polar (e.g. Gly) or polar (e.g. Ser or Thr) amino acids and include “GS” linkers consisting of stretches of Gly and Ser residues. An example of the most widely used flexible linker which is also envisioned for the conjugate of the present invention has the sequence of (Gly-Gly-Gly-Gly-Ser)n. Treatment

The term “treatment” in all its grammatical forms includes therapeutic or prophylactic treatment of B-cell malignancies. A “therapeutic or prophylactic treatment” comprises prophylactic treatments aimed at the complete prevention of clinical and/or pathological manifestations or therapeutic treatment aimed at amelioration or remission of clinical and/or pathological manifestations. The term “treatment” thus also includes the amelioration or prevention of B-cell malignancies.

Preferably, treatment involves administration of a therapeutically effective amount of BCR antigen conjugate. By “therapeutically effective amount” is meant an amount of the BCR antigen conjugate that elicits a therapeutic effect as described herein. The exact dose of BCR antigen conjugate will depend on the purpose of the treatment (e.g. remission maintenance vs. acute flare of disease or metastatic spread), and will be ascertainable by one skilled in the art using known techniques. Adjustments for route of administration, age, body weight, general health, sex, diet, time of administration, drug interaction and the severity of the condition may be necessary, and will be ascertainable with routine experimentation by those skilled in the art.

In the context with the present invention the term “therapeutic effect” in general refers to the desirable or beneficial impact of a treatment, e.g. amelioration or remission of the disease manifestations. The term “manifestation” of a disease is used herein to describe its perceptible expression, and includes both clinical manifestations, hereinafter defined as indications of the disease that may be detected during a physical examination and/or that are perceptible by the patient (i.e., symptoms), and pathological manifestations, meaning expressions of the disease on the cellular and molecular level. E.g., the general appearance of the respective patient (e.g., fitness, well-being) can be evaluated which will also aid the skilled practitioner to evaluate whether a therapeutic effect has been elicited. The skilled person is aware of numerous other ways which are suitable to observe a therapeutic effect of the BCR antigen conjugate of the present invention.

The term “patient”, also termed “subject” herein, is intended to include any mammal or any vertebrate that may be used as a model system for human disease (i.e., B-cell malignancy). Examples of subjects include humans, monkeys, apes, dogs, cats, mice, rats, horses, pigs, cows, sheep, goats, rabbits, guinea pigs, hamsters and transgenic species thereof. Preferred in the context of the present invention are human patients.

A variety of routes are applicable for administration of the conjugate of the present invention, including, but not limited to, orally, topically, transdermally, subcutaneously, intravenously, intraperitoneally, intramuscularly or intraocularly. However, any other route may readily be chosen by the person skilled in the art if desired.

### Combination Therapy

The BCR-antigen conjugate of the present invention is also envisaged for combination therapy together with common therapeutic approaches used for the treatment of B-cell malignancy. Therapeutic approaches conceivable for combination therapy includes, e.g., CHOP (cyclophosphamide, doxorubicin, vincristine, and prednisone), optionally completed by rituximab (R-CHOP), BR (bendamustine and rituximab), R-CVP (rituximab, cyclophosphamide, vincristine, prednisone), and fludarabine-based combinations. Radiation therapy (typically external-beam radiation therapy) is also envisaged, as treatment with monoclonal antibodies like rituximab, ofatumumab, obinotuzimab, or the antibody-drug conjugate brentuximab vedotin. A stem cell transplant may also be required. Additional administration of palliative agents in order to reduce or control side effects of B-cell malignancy treatment and symptoms, including pain, nausea, breathlessness, insomnia, and other physical symptoms caused by the B-cell malignancy or its treatment is also envisaged.

The skilled person will acknowledge that the treatment options vary depending on the specific B-cell malignancy to be treated. In general, treatment with the BCR antigen conjugate of the invention may precede and/or antecede common therapeutic approaches, or may be conducted simultaneously. Although it is envisaged herein to combine the conjugate of the invention with common therapeutic options, it may as well be used to replace components of the chemotherapeutic regimens commonly used to treat B-cell malignancies. For example, due to its superior specificity and, hence, expected reduced side effects, it is also envisioned that the conjugate of the invention may be used to replace rituximab or vincristine treatment (e.g. in R-CHOP).

### Diagnostic Method

The use of BCR antigen conjugates in diagnostic methods is also contemplated. Said diagnostic methods are based an identification and characterization of malignant B-cells expressing a BCR recognizing the BCR antigen conjugate in a subject, and can be conducted in vivo, ex vivo or in vitro. In vivo methods may include administering to the subject a labeled BCR antigen conjugate. For ex vivo or in vitro methods, a body fluid, tissue or cell sample, in particular a blood sample, suspected of containing malignant B-cells binding to the conjugated BCR antigen of the invention can be contacted with a labeled BCR antigen conjugate capable of binding to BCR of said cells. Binding of the labeled conjugate indicates the presence of cells expressing a BCR having a BCR specifically recognizing the antigenic part of the conjugate in the sample, and can be detected by methods well known to those of skill in the art. Some aspects of the invention relate to diagnosing or monitoring a B-cell malignancy in a subject by determining the presence or amount or level of malignant B-cells recognizing the conjugated BCR antigen. The presence or level of such B-cells may be determined using routine methods known to those of skill in the art. In particular, a (labeled) BCR antigen conjugate may help in supporting the suspicion of a certain B-cell malignancy previously diagnosed based on conventional diagnostic methods.

### Stratification

Also envisaged herein is a non-invasive method for identifying a patient suffering from a B-cell malignancy as described herein, said patient being disposed to respond favorably to a conjugated BCR antigen of the invention. Said method comprises: (i) providing a labeled BCR antigen conjugate, (ii) adding the labeled BCR antigen conjugate to a sample of each patient comprising malignant B-cells; (iii) assessing binding and/or internalization of the labeled BCR antigen conjugate to said malignant B-cells as described herein.

The term “identifying a patient disposed to respond favorably to the conjugated BCR antigen of the invention includes stratifying patients. The term “stratifying” refers to sorting patients into those who may or may not benefit from therapy with said conjugate. In particular, stratifying patients involves determining the capability of the patient's malignant B-cells to bind to and/or internalize the conjugate of the invention. Patients whose malignant B-cells bind to and/or internalize the labeled BCR antigen conjugate are herein defined as being disposed to react favorably to treatment with the conjugated BCR antigen, whereas patient whose malignant B-cells do not bind and/or internalize the labeled BCR antigen conjugate are not.

“Disposed to respond favorably” means that a patient is likely to experience a beneficial effect when treated with the conjugated BCR antigen of the invention. Beneficial effects include inhibition and/or killing of malignant B-cells, reduction in the number of malignant B-cells, and/or a therapeutic effect as ascertainable using routine methods known in the art. Preferably, said patient has previously been diagnosed with a B-cell malignancy.

### Composition

It is envisaged to administer the BCR antigen conjugate in the form of a pharmaceutical composition. Preferably, said BCR antigen conjugate is present in the pharmaceutical composition in a therapeutically effective amount. The term “pharmaceutical composition” particularly refers to a composition suitable for administering to a human, i.e., a preferably sterile composition containing components which are pharmaceutically acceptable. Preferably, a pharmaceutical composition comprises a BCR antigen conjugate together with one or more pharmaceutical excipients. The term “excipient” includes fillers, binders, disintegrants, coatings, sorbents, antiadherents, glidants, preservatives, antioxidants, flavoring, coloring, sweeting agents, solvents, co-solvents, buffering agents, chelating agents, viscosity imparting agents, surface active agents, diluents, humectants. carriers, diluents, preservatives, emulsifiers, stabilizers or tonicity modifiers. Pharmaceutical compositions of the invention preferably comprise a therapeutically effective amount of the BCR antigen conjugate and can be formulated in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in any other form which is particularly suitable for the desired method of administration.

The pharmaceutical composition of the present invention may further comprise one or more additional agents. Preferably, said agents are therapeutically effective for treatment of B-cell malignancies. Particular additional agents that can be used in the pharmaceutical composition of the invention include, without limitation, toxins as described elsewhere herein, including chemotherapeutic agents, or palliative agents.

It is to be noted that all aspects described in the context of the BCR antigen conjugates or kit comprising the same, and methods of treatment are also applicable to the pharmaceutical composition of the invention, mutatis mutandis.

In view of the foregoing, the invention also provides for the use of the B-cell antigen conjugate as defined herein for the manufacture of a pharmaceutical composition for treatment of B-cell malignancies.

### Kit

It is also envisaged that the BCR antigen conjugate can be used as part of a kit. Accordingly, in a further aspect, the present invention also relates to a kit comprising a BCR antigen conjugate, which is in particular intended for use in a method of treatment of B-cell malignancies as defined elsewhere herein.

The kit may be a kit of two or more parts, and comprises the BCR antigen conjugate, or a pharmaceutical composition comprising said BCR antigen conjugate, and one or more additional agents as defined in the context of the pharmaceutical composition. The components of the kit may be contained in a container or vials. It is to be noted that all aspects described in the context of the BCR antigen conjugates or pharmaceutical compositions comprising the same, and methods of treatment are also applicable to the kit of the invention, mutatis mutandis.

It is envisaged that the additional agents comprised in the kit may be applied simultaneously, or sequentially, or separately with respect to the administration of BCR antigen conjugate. The present invention further encompasses the administration of the agents via different administration routes.

Another aspect of the present invention is a method of treatment of B-cell malignancies in a subject in need thereof, comprising administering a therapeutically effective amount of a B-cell antigen conjugate as defined herein to said subject. The person skilled in the art will acknowledge that all aspects described herein in the context the BCR antigen conjugate, the pharmaceutical composition and the kit comprising the same are applicable to the method of treatment, mutatis mutandis.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be had from the following examples, offered for illustrative purposes only. The examples are not intended to limit the scope of the present invention in any way.

### EXAMPLES

### Example 1

The present inventors have developed approaches for clinical use of BARs for a variety of B-cell malignancies as set out in the following:

### Chronic Lymphocytic Leukemia (CLL):

The incidence of CLL is estimated to be 4 new cases per 100 000 persons per year. There are about 14 620 new cases of CLL and 4650 deaths from CLL each year in the US (American Cancer Society 2014: Cancer Facts and Figures 2014; retrieved from http://www.cancer.org/acs/groups/content/@research/documents/webcontent/acspc-042151.pdf), and 10 000 deaths across Europe each year 7.

Clinical indications for BARs: CLL is a slowly evolving disease which is treated when symptoms occur. With modern first-line treatments, the responses are frequent, providing most patients with prolonged treatment-free periods. For patients necessitating retreatment, many different regimens are an option, and many patients receive up to a dozen treatments before they succumb to the disease. However, as with multiple myeloma CLL is believed to be incurable without allogeneic transplantation which is rarely possible in this mostly elderly population. The choice of sequences of the different available regimens is based on efficacy and tolerability and there is still space for new drugs with high efficacy and low toxicity, features that can be assumed for BARs. While to date BARs are available for ca. 30% of all CLL cases, the problem with BARs for CLL is that the antigenic BCR targets are quite diverse, with most of them having different epitopes within a given BAR in cases with a known BAR. Despite the expected low costs for producing BARs it is doubtful that there will be a wide clinical application of BARs, if an individual license procedure is necessary for any of the available CLL BARs. A group-wide license for CLL-BARs should be pursued.

### Identification of Candidate Patients:

Test peripheral blood (CLL cells) for BCR specificity by a well-established whole-blood lysate ELISA using the known antigens as a coat.

### Implementation Strategy:

Demonstrate single-drug activity of CLL-BARs in (multiply) relapsed CLL patients (in theory of the 15 000 CLL deaths per year in the Western world, 30% of whom would be potential candidates for such a phase-I/II study.

If active, in a next step randomize CLL patients responding to first-fine treatment into maintenance BARs or observation.

As a third step (alternatively after step 1): randomize patients for first-line treatment with and without BARs (induction plus maintenance).

### Mantle Cell Lymphoma (MCL)

MCL comprise ca. 3% of all cases of non-Hodgkin lymphoma. The overall incidence in the US is 0.55 per 100 000 per year and increases with age: 0.07 in patients aged <50 years, 2.97 in patients aged 70 to 79 years, and 2.78 in those aged ≥80 years. The incidence of MCL is higher in men (0.84 of 100,000) than in women (0.34 of 100,000; P<0.05), and higher in Caucasians (0.61 of 100,000) than in African Americans (0.32 of 100,000). Late-stage (III-IV) MCL is diagnosed in 74.6% of patients 8. About 4200 new cases of MCL per year are estimated for the US and 6000 in Europe. Currently, MCL is treated using combination chemotherapy or chemotherapy plus immunotherapy, followed by stem cell transplantation. However, agents such as bortezomib, ibrutinib and lenalidomide have recently demonstrated an ability to improve progression-free survival (PFS) and overall survival (OS) by targeting B-cell receptor signaling suggesting a significant improvement from the previous 3-year survival rate to a median of 7 years. Response rates to initial combination chemotherapy regimens are often very good in patients with MCL; however, relapse is very common, and it is unclear if patients can be cured by the available treatment options yet, perhaps with the exception of allogeneic transplantation which is feasible in only a minority of these mostly elderly patients with a median age of diagnosis of 70 years 9. Therefore, new approaches are badly needed.

Clinical indications for BARs: Despite considerable improvement of treatment results achieved by new drugs in recent years, the cure rate of MCL remains low, and few patients are cured by first line treatment. Therefore, MCL remains an unmet medical need. A BAR (LRPAP1) has been identified in 4/9 (44%) of cases with MCL. The high percentage of patients with MCL who are potential candidates for BAR treatment should accelerate the clinical development of MCL-BARs.

### Identification of Candidate Patients:

Check MCL-BCR for binding to LRPAP-1. Test using peripheral blood under development.

### Implementation Strategy:

Demonstrate single-drug activity of MCL-BARs in relapsed MCL patients in a phase-I/II trial. Due to the expected high activity and low toxicity of MCL-BARs this could be done in first relapse of elderly, and second relapse of younger MCL patients. Of the estimated 10 000 new MCL cases every year in the Western world, 4400 are expected to have a BCR specific for LRPAP1. Of these, one quarter can be expected to relapse each year; therefore an estimated 1000 patients would be potential candidates for such a phase-I/II trial per year.

If active, in a next step randomize MLL patients responding to first-fine treatment into maintenance BARs or observation.

As a third step (alternatively after step 1): randomize patients for first-line treatment with and without BARs (induction plus maintenance).

### Primary Central Nervous System Lymphoma (PCNSL)

PCNSL represents 3.1% of all primary brain tumors and 2-3% of NHLs. After a three-fold rise observed two decades ago, the incidence of PCNSL has increased only slightly in the past 10 years in individuals above the age of 60, and now stands at 0.46 per 100,000 patient-years. 1000 new patients are estimated for the USA per year. Median age at diagnosis is 60-65 years, and median survival is 10-20 months, with survival of <20%-30% at 5 years 10. Patients older than 60 years do substantially worse, with a 5-year survival rate of 19%, compared with 75% for younger patients. High-dose chemotherapy with autologous stem cell transplantation might improve the results, but is not feasible in all patients 11, 12. Prognosis at relapse is dismal, particularly if the lymphoma is resistant to high-dose chemotherapy.

Clinical indications for BARs: Two thirds of all PCNSL cases have a BCR with specificity for the SAMD14 domain of neurabin-1, a protein preferentially expressed in the CNS. Interestingly, in these patients Neurabin-1/SAMD14 is hyperglycosylated compared to healthy controls. The reason for this hyperglycosylation is unknown, but supports the hypothesis that this modified protein induces a chronic antigenic response of B-cells with specificity for neurabin-1, ultimately resulting in the clonal evolution of a malignant clone. Because of the poor prognosis of PCNSL, in particular of relapsing PCNSL, there is an unmet need for more effective treatment. Therefore, despite of the rarity of PCNSL, the clinical development of PCNSL-BARs should proceed quickly, also because ⅔ of all patients with PCNSL are potential candidates for treatment with PCNSL-BARs. Of the estimated 2000 new cases of PCNSL in the Western world per year, at least one quarter should be available per year for phase-I/II studies. A prerequisite for PCNSL-BARs is that they penetrate the blood-brain barrier. With s small molecule like a BAR representing only the BCR-binding epitope it can be expected that it penetrates the blood-brain barrier, and the small size of a BCR-binding epitope should leave room for several pharmacologic modifications aiming at the breakdown of the blood brain barrier

### Identification of Candidate Patients:

Test peripheral blood for hyperglycosylated neurabin by established ELISA.

### Implementation Strategy:

Demonstrate single-drug activity of PCNSL-BARs in relapsed PCNSL patients in a phase-I/II trial. Due to the dismal prognosis of relapsed patients a phase I/II study with 50 patients should suffice to show a strong signal who should be recruitable within 1 year.

If active, in a next step PCNSL-BARs should be combined with standard salvage therapy regimens in a randomized phase-II study.

As a third step (or in parallel with step #2) a randomized phase-III study of standard treatment with and without PCNSL-BARs should be performed. Aim should be an improvement of progression-free survival by 6 months.

### Diffuse Large B-Cell Lymphoma (DLBCL)

Diffuse large B-cell non-Hodgkin's lymphoma (DLBCL) is the most frequent non-Hodgkin lymphoma and constitutes (depending on the geographic region 30%-58% of non-Hodgkin's lymphoma series. The crude incidence in the European Union is 3-4/100 000/year. The incidence increases with age from 0.3/100 000/year (35-39 years) to 26.6/100 000/year (80-84 years). The incidence of NHL increased dramatically from the 1970s until the middle of the 1990s with an estimated 65,540 new cases expected in the United States in 2010, thus making NHL the seventh most common cancer (excluding basal cell and squamous cell skin cancers) 13. During this period, the steady increase of 3% to 4% per year in incident cases of lymphoma and DLBCL occurred in both sexes, across racial categories, and across all age groups except the very young. Incidence rates plateaued in the late 1990s, and from 1992 to 2006, the age-adjusted incidence rate for DLBCL increased approximately 1% per year, from 8.07 to 8.98 cases per 100,000 population. The increase in DLBCL incidence from the 1970s represents an unprecedented rise comparable only to the rise in skin cancer.

The prognosis of DLBCL patients has improved considerably since the introduction of the monoclonal antibody rituximab, and 75% of the patients can be expected to be cured by a combination immunochemotherapy consisting of rituximab and CHOP (cyclophosphamide, doxorubicin, vincristine and prednisone. The cure rate depends on the number of risk factors according to the International Prognostic Index (IPI): age >60 year, elevated serum levels of pre-treatment LDH, advanced stage (III/IV according to the Ann Arbor staging system) and >1 extralymphatic site of involvement. Besides the IPI, other independent risk factors have been identified, one of the most widely accepted being the cell of origin (COO). According to the COO, DLBCLs derived from the germinal center (GC type) are distinguished from DLBCLs derived from an activated B-cell (ABC type), leaving a minor group of cases to which no COO can be assigned. The ABC type is estimated to represent ca. 40% of all DLBCL and its frequency is increasing with age, with most series looking at patients >60 years of age observing at least as many ABC-as GC-DLBCL. While the cure or 5-year survival rate of GC-DLBCL as determined by gene expression profiling was reported to be 75%, it was only 40% for ABC-DLBCL 14. Other subtypes for which a poor outcome has been reported are DLBCL with a MYC break, BCL2 break, double-hit DLBCL and DLBCL expressing both MYC and BCL2 protein.

Clinical indications for BARs: ⅗ DLBCL cell lines of the ABC-type expressed BCR receptors with specificity for the ARS2 protein, but none of 4 DLBCL cell lines of the GC type. Interestingly, in these patients ARS2 (as in the DLBCL cell lines) was hypophosphorylated. The reason for this hypophosphorylation is unknown, but supports the hypothesis that this modified protein induces a chronic (auto-)antigenic stimulation of B-cells with specificity for ARS2, ultimately resulting in the clonal evolution of a malignant clone. Ex-vivo or biopsy data from DLBCL are still limited, but it appears that the DLBCL of the ABC subtype do not express BCR with specificity for ARS2 as frequently as the respective cell lines. However, since nearly all of the >100 000 new DLBCL cases in the USA and Europe per year are treated and one third of them will either not achieve a response or relapse after achieving a response, ca. 33 000 of them can be calculated to need salvage treatment. In sharp contrast to the results of first-line treatment, treatment results of relapsed DLBCL have become considerably worse since the introduction of rituximab. Despite salvage treatment including high-dose chemotherapy and autologous stem-cell transplantation only 20% of young patients achieve durable second remissions and the rate of durable remissions is even lower in relapsing (elderly patients) who are not fit enough for high-dose chemotherapy. A recent study of fresh biopsies from ABC-DLBCL showed that 8/31 expressed hypophosphorylated ARS2. Since relapsed DLBCL are enriched for the ABC subtype of DLBCL a prevalence of 10% to 20% of all relapsed DLBCL can be assumed to expressed hypophosphorylated ARS2, hence >3000 patients can be expected per year for whom DLBCL-BARs would be an option.

### Identification of Candidate Patients:

Test peripheral blood for hypophosphorylated ARS2 by established ELISA.

### Implementation Strategy:

Demonstrate single-drug activity of DLBCL-BARs in relapsed DLBCL in a phase-I/II trial. Due to the high prevalence of these patients, the recruitment of 50 patients into a phase I/II should be feasible within 1 year.

If active, in a next step DLBCL-BARs should be combined with standard salvage therapy regimens in a randomized phase-II study. Goal should be a prolongation of PFS by 6 months.

As a third step (or in parallel with step #2) a randomized phase-III study of standard treatment with and without DLBCL-BARs should be performed. Aim should be an improvement of progression-free survival by 7.5 to 10%.

### Follicular Lymphoma (FL)

Follicular lymphoma (FL) is the second most common lymphoma in the United States, representing nearly 14,000 patients diagnosed annually 13 with an incidence of 3 cases/100 000 per year. The estimated incidence in Europe is 2.18 cases per 100,000 persons per year 15 so that a similar number of new cases can be expected in USA and Europe. The incidence is stable over time, but varies by ethnicity with the incidence in Whites being more than twice that in Black and Asian populations 16-18.

Despite the advanced stage, the median survival ranges from 8 to 15 years, leading to the designation of being indolent. Watchful waiting, i.e., the deferring of treatment until the patient becomes symptomatic, is an option for patients with advanced-stage follicular lymphoma Patients with advanced-stage follicular lymphoma are not cured with current therapeutic options. Even though >90% of the patients respond to first-line regimens (CHOP, bendamustine), the rate of relapse is fairly consistent over time, even in patients who have achieved complete responses to treatment 19 and the 5-year progression-free survival is ca. 50% 20.

Clinical indications for BARs: Though not systematically and consequently investigated, the only BAR identified to date is optineurin, which was found to be the target of the BCR of 4/40 patients with follicular lymphoma. Though 50% of patients need retreatment with 5 years, there are many treatment options and patients usually respond to multiple salvage treatments. With only 10% of FL patients being potential candidates for treatment with an FL-BAR, finding an indication for BARs in this lymphoma type might be difficult.

### Identification of Candidate Patients:

Test to be established.

### Implementation Strategy:

Demonstrate single-drug activity of FL-BARs in multiple relapsed FL patients in a phase-I/II trial. Due to the low rate of available FL-Bars to date, the recruitment of 50 patients into a phase I/II might take several years. A response rate of 30% would be a clinically relevant goal

If active, a randomized phase-III study in relapsing patients combining FL-BARs with a popular salvage treatment would be an option.

Another option would be a randomized study of FL-BARs for consolidation/maintenance in patients responding to first-line treatment, either in all responding patients or only in those responding patients were minimal residual disease can be detected an monitored.

### Example 2: Internalisation of ARS2 in U2932 Cells

After incubation with FLAG-tagged ARS2 (Uniprot Acc. No. Q9BXP5, version 127 last modified Apr. 13, 2016) protein (10 μg/ml, 30 min, 4° C.) the amount of cells was divided into 2 pools of which the first one was kept at 4° C. and stained immediately, while the other one was incubated for 1 h at 37° C. before staining was done. Both pools were stained at 4° C. by the following procedure: each pool was subdivided into 2 parts of which one part was kept untreated while the other one was treated with 2% PFA/0.2% saponin. Then mouse anti-FLAG mAb was added (30 min) followed by intense washing (PBS) and incubation with anti-mouse-FITC mAb (30 min). Thereafter, cells were washed again (PBS 4° C.) and analyzed by FACS.

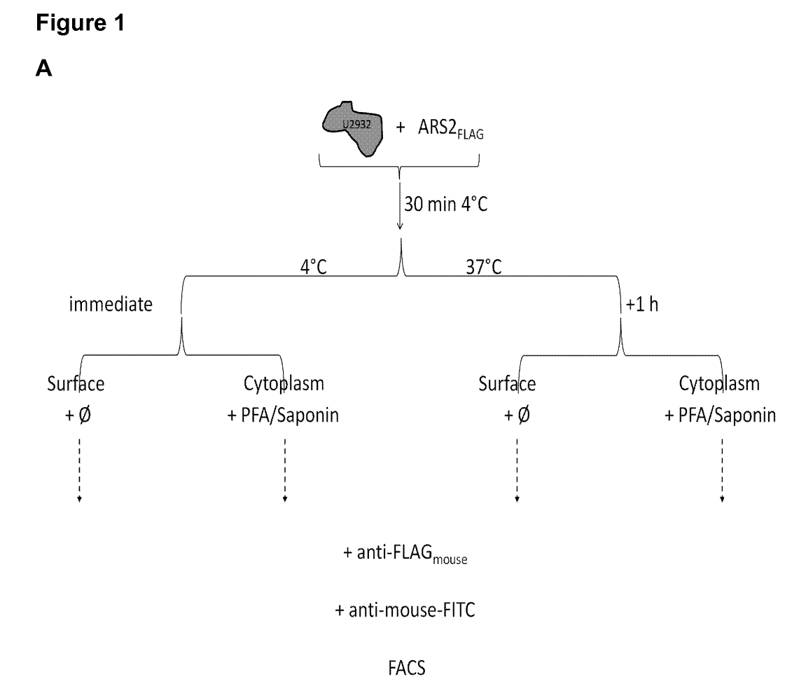

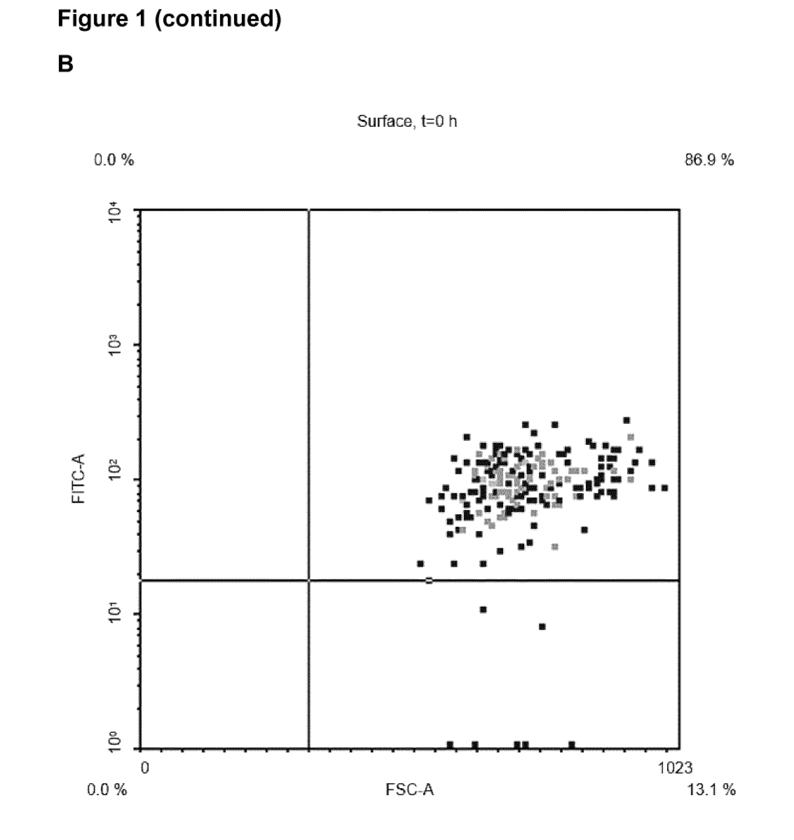

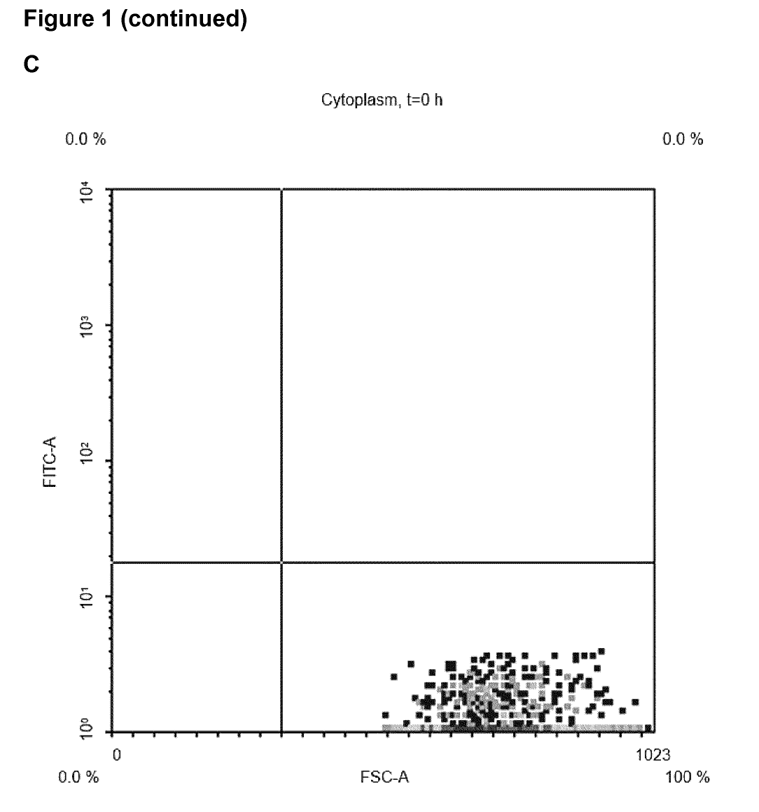

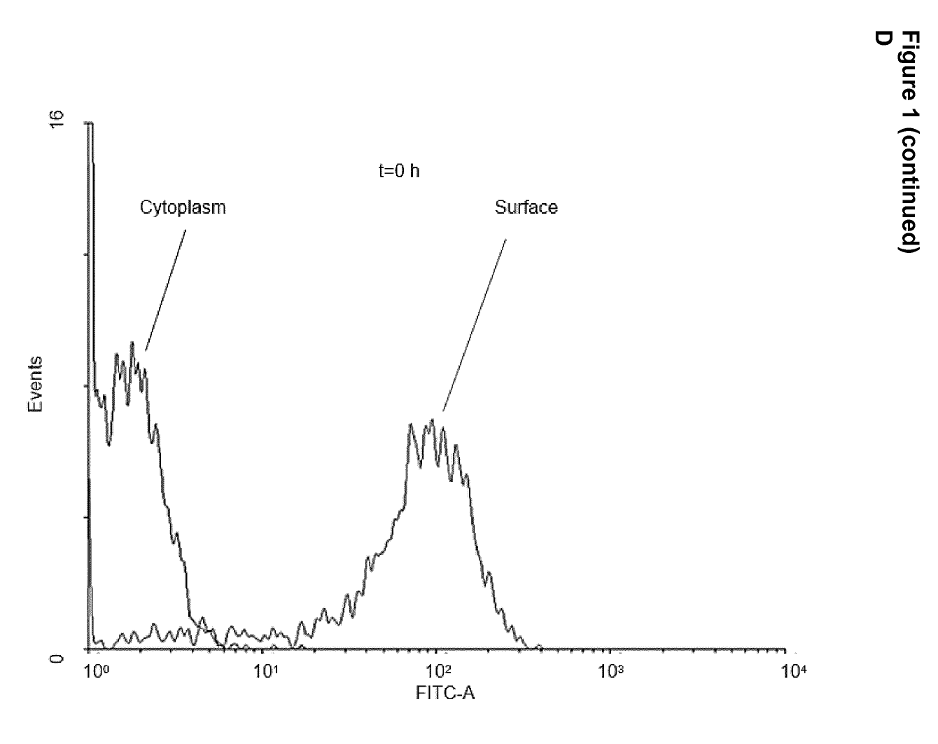

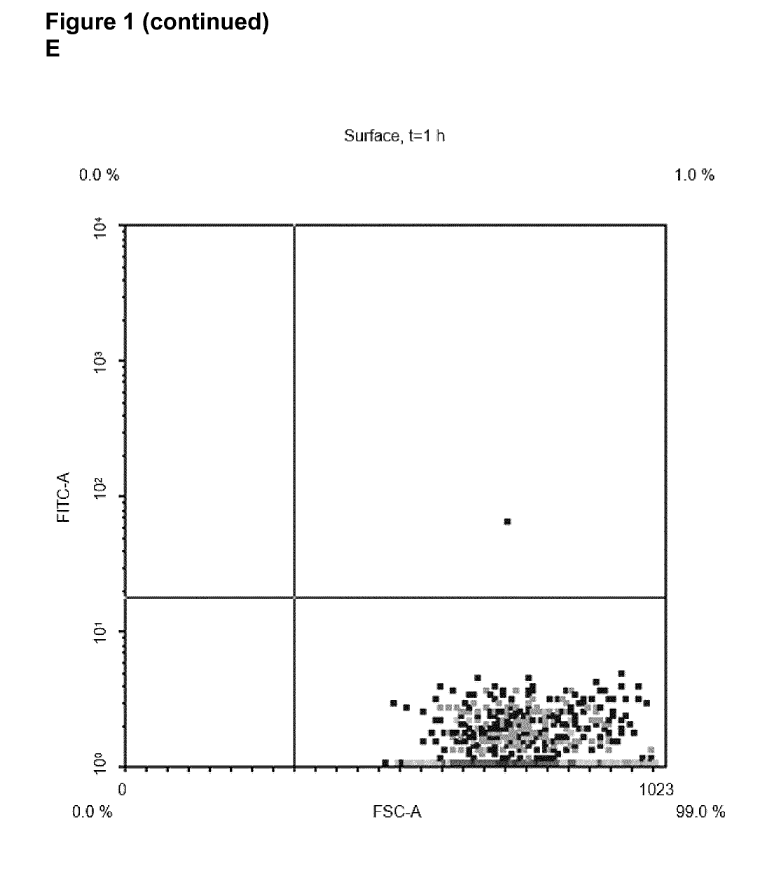

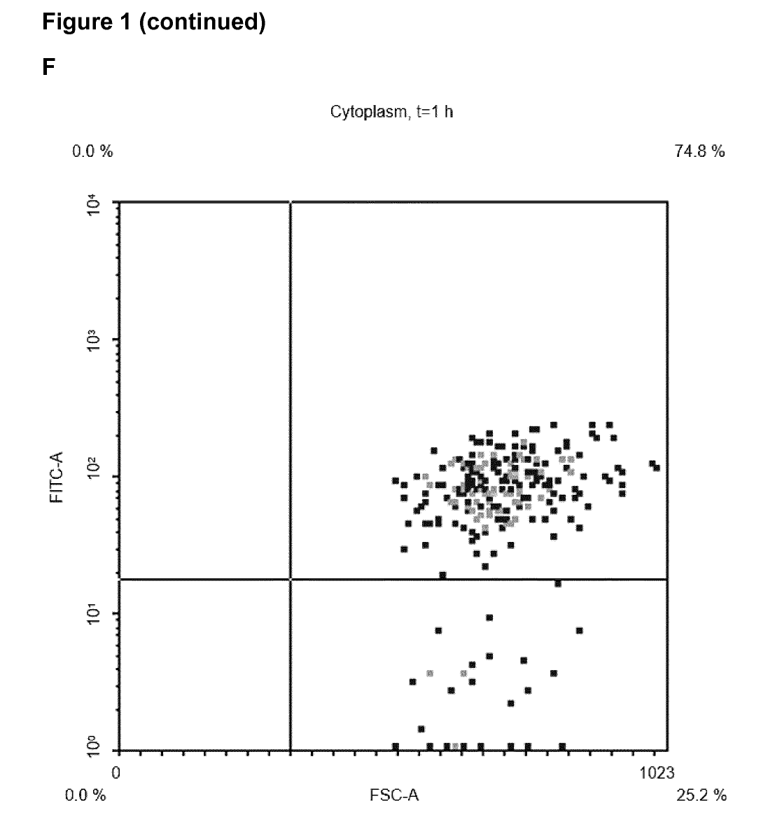

FACS analysis clearly shows that at 4° C. ARS2 was only detected at the surface of the cells, while ARS2 was enriched in the cytoplasm after internalization after 1 h at 37° C. X-axis: FSC; Y-axis: FITC signal (FIG. 1)

### Example 3: Inhibition of Proliferation of Immunotoxin-Treated ABC-Type DLBCL Cells

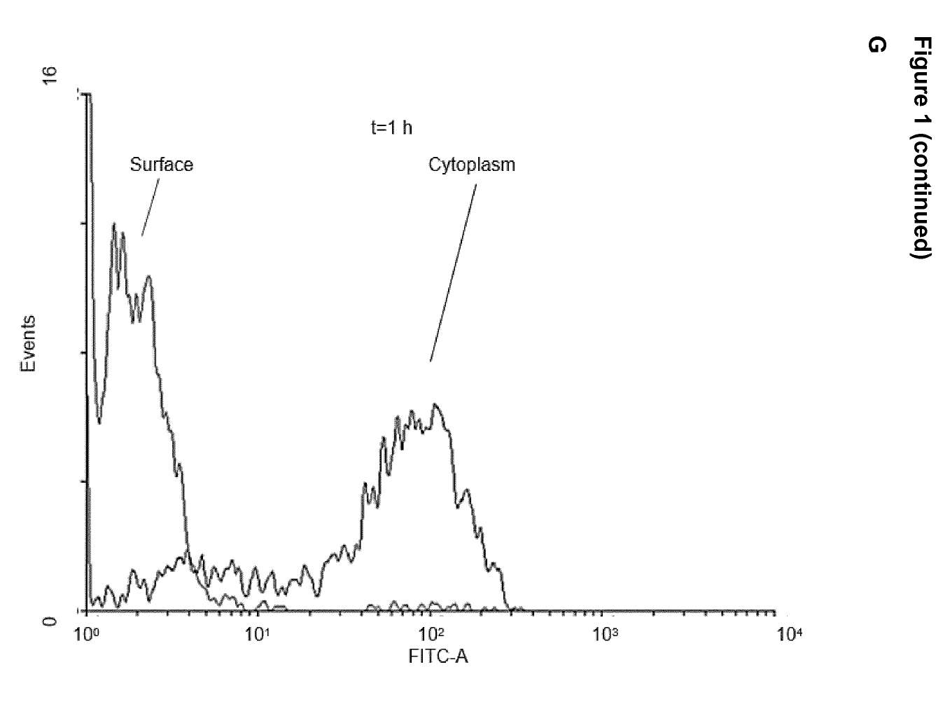

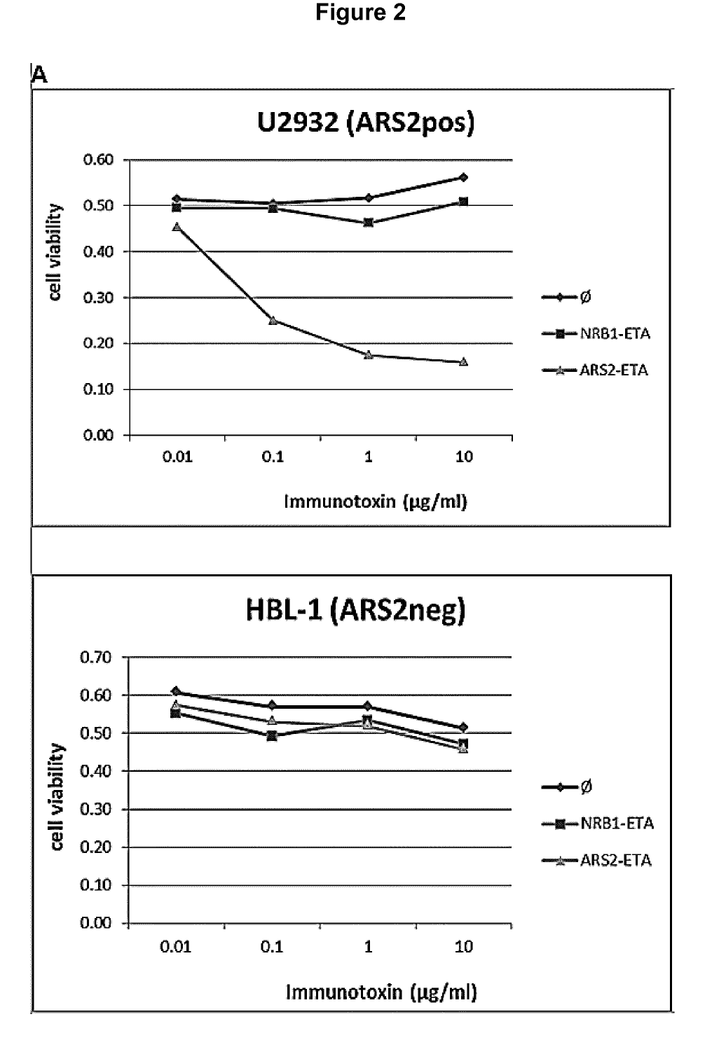

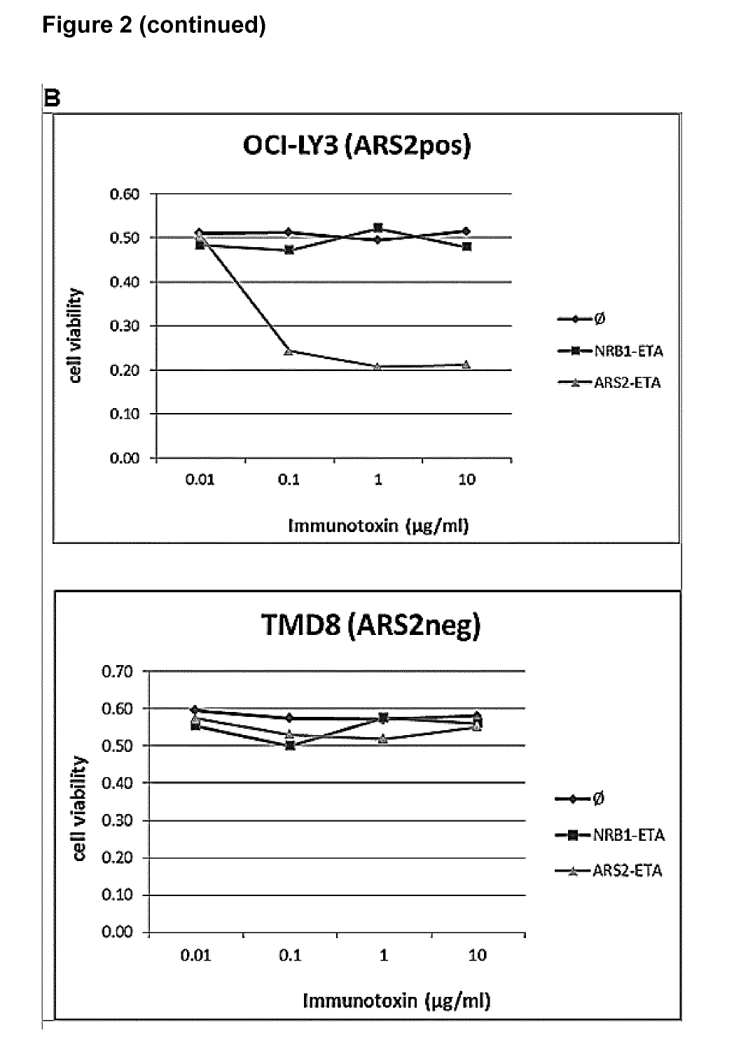

Inhibition of proliferation of immunotoxin-treated ABC-type DLBCL cells. ARS2 positive (U293, OCI-Ly3) and ARS2 negative cells lines (HBL-1, TMD8) were cultured (37° C., 24 h) in the presence of pseudomonas exotoxin coupled to ARS2 (ARS2-ETA) or neurabin (NRB1-ETA) as control. Cell proliferation was measured using the EZ4U kit (Biomedica, Vienna, Austria) according to the manufacturer's instructions (FIG. 2)

### Example 4: Killing of OCI-Ly3 Cells by ARS2-Immunotoxin Construct

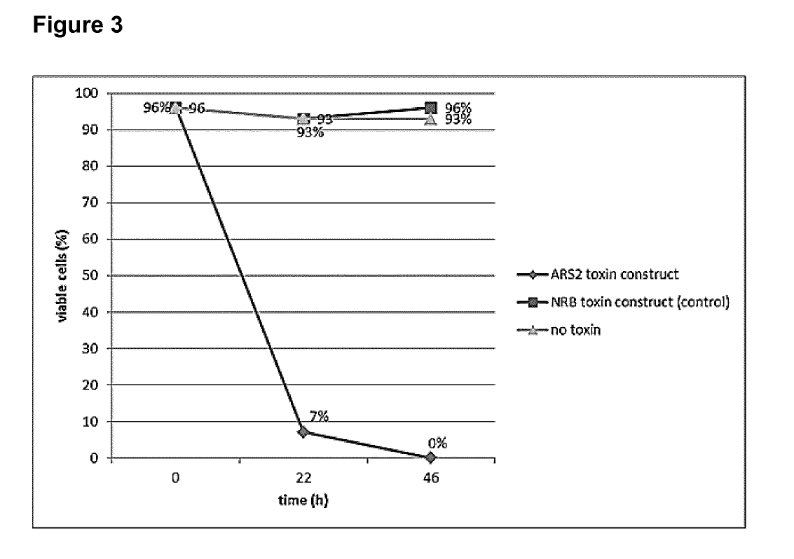

OCI-Ly3 cells were cultured in the presence of ARS2-ETA or NRB1-ETA (2.5×104 cells/1 ug toxin/well). Cultures were analysed for viable cells by staining with trypan blue and counting (FIG. 3).

### Example 5: Specific Inhibition of Growth in SCID Mice of Heterotransplanted ARS2-Positive Human DBLC Cells of the ABC Type by Toxin-Conjugated ARS2

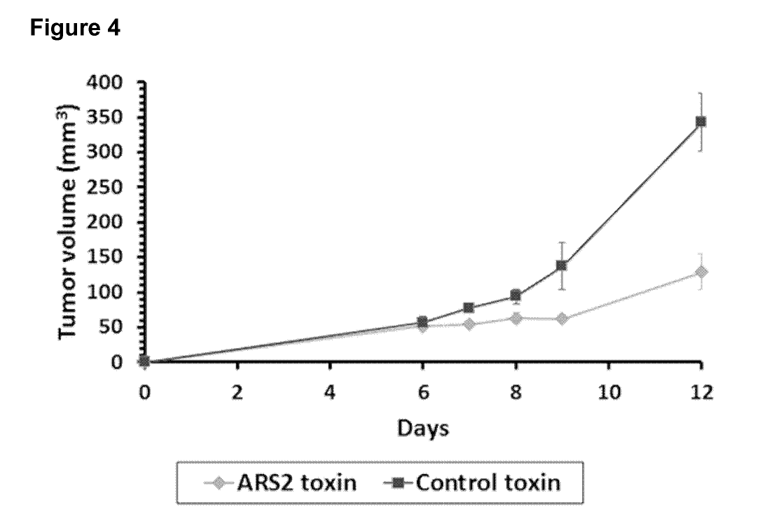

5-7 week old female SCID-beige mice were obtained from Charles River Laboratories (Köln, Germany). 5×10⁶ ARS2-positive OCI-LY3 cells in 100 μl PBS/matrigel each were injected on day 0 subcutaneously into the flanks of 5-7 week-old female SCID beige mice. 200 μl of the ARS2-ETA or NRB1-ETA as a control, respectively, at a dosis of 75 μg toxin/ml in PBS were injected once on day 6 into the tail vein on day 6 after tumor inoculation. The tumor diameter was measured daily in two vertical directions using a sliding caliper. Tumor volume was calculated according to V=(π/6)×smallest diameter×tallest diameter. Results from 3 ARS2-ETA (green line) and ARB1-ETA treated mice are shown in FIG. 4.

### Example 6: Binding of CD3-ARS2 to DLBCL Cell Lines

### Background

Autoantigens are suspected to play an important role in the genesis of different types of B cell neoplasia. Suggestive of this hypothesis is the restricted usage of a stereotyped IgHv repertoire in CLL, MCL and as most recently discovered in DLBCL. ARS2 was recently identified as growth stimulating autoantigen for approximately 5% of DLBCLs, here termed ARS2 reactive lymphomas, which binds with high affinity to the B cell receptor (BCR) of a significant portion of DLBCLs. We designed a bispecific product containing a recombinant single chain fragment (scFv) against CD3 linked to ARS2 (CD3-ARS2). One site engages the T cell co-receptor CD3 of human T cells, and the other site recognizes with high specificity the B cell receptor of ARS2 reactive lymphomas in order to redirect T cells to lymphoma cells. We tested this bispecific construct for selective cytotoxicity of ARS2 reactive lymphoma cell lines.

### Material and Methods

VL and VH genes of the OKT 3 hybridoma and the DNA sequence of the 33 amino acids containing epitope of ARS2 were cloned in a pcDNA 3.1 vector by standard cloning techniques. VH and VL were linked by a glycine-serine linker, as was VL to the ARS2 epitope resulting in VH-GlySer-VL-GlySer-ARS2 peptide chain. A histidine tail was included for later detection and purification.

The final cloning product was transfected in HEK cells for production of the bispecific construct. Binding capacity to lymphoma cell lines (OCI-Ly 3, U2932, HBL-1) and PBMCs was assessed by flow cytometry. Western blot analysis was used for detection of CD3-ARS2 after incubation with the monoclonal anti-His Tag antibody. Cytotoxicity was evaluated by LDH release assay.

Transfection in HEK 293 cells was done using X-tremeGENE™ HP DNA Transfection Reagent by Sigma-Aldrich. Binding capacity to lymphoma cell lines (OCI-Ly 3, U2932, HBL-1) and PBMCs was assessed by flow cytometry. Western blot analysis was used for detection of CD3-ARS2 after incubation with the monoclonal anti-His Tag antibody. Purification of the protein was accomplished using cobalt-based “Immobilized Metal Affinity Chromatography (IMAC)”. This technique uses magnetic beads which bind to histidine-tagged proteins, e.g. CD3-ARS2. Cytotoxicity was evaluated with a LDH release assay (“Cytotoxicity Detection Kit” by Roche).

Primers used were as follows:

CD3-VH-HindIII (5′ - 3′):
AAGCTTGCCACCATGCAGGTCCAGCTGCAGCAG
CD3-VH-BamHI (5′ - 3′):
GGATCCACCACCACCGGAGCCGCCGCCGCCAGAACCACCACCACCAGAAC
CACCACCACCTGTTGTTTTGGCTGAGGA
CD3-VK-BamHI (5′ - 3′):
GGATCCCAAATTGTTCTCACCCAGTC
CD3-VK-EcoRI (5′ - 3′):
GAATTCGATCCGCCACCGCCAGAGCCACCTCCGCCTGAACCGCCTCCACC
AGTTGGTGCAGTATCAGCC
ARS2 AA342-EcoRV (5′ - 3′):
GATATCATGAAGGAAGCCAAAAAGAGTAGC
ARS2 AA375-His-SmaI (5′ - 3′):
CCCGGGTTAATGGTGGTGGTGATGATGAGCGCTCTCTGACTCCGACTCAG
AC

### Results

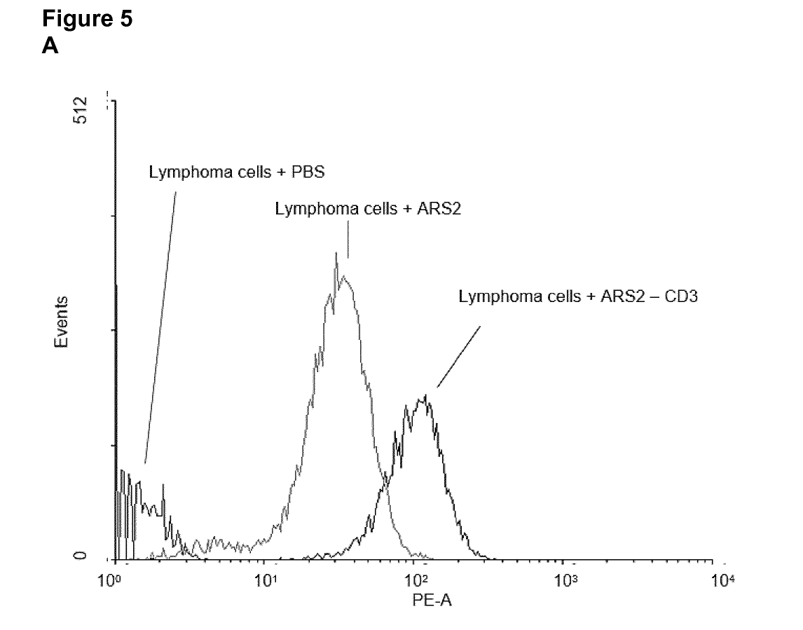

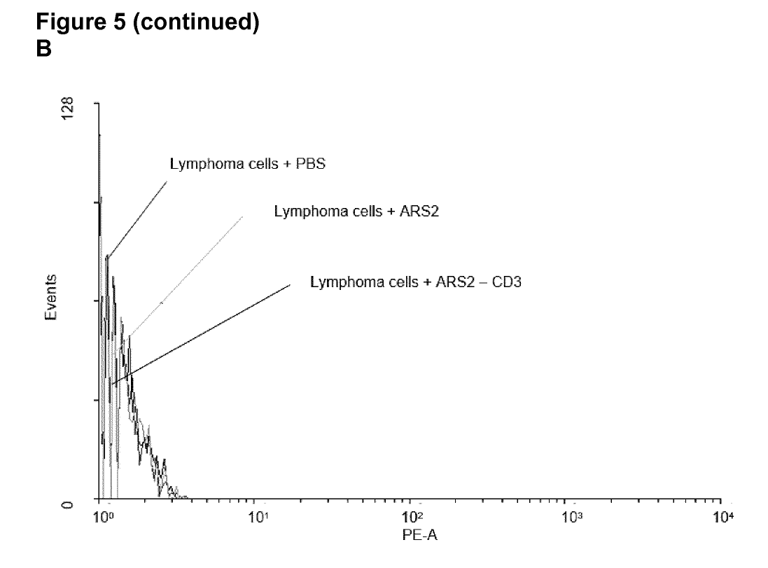

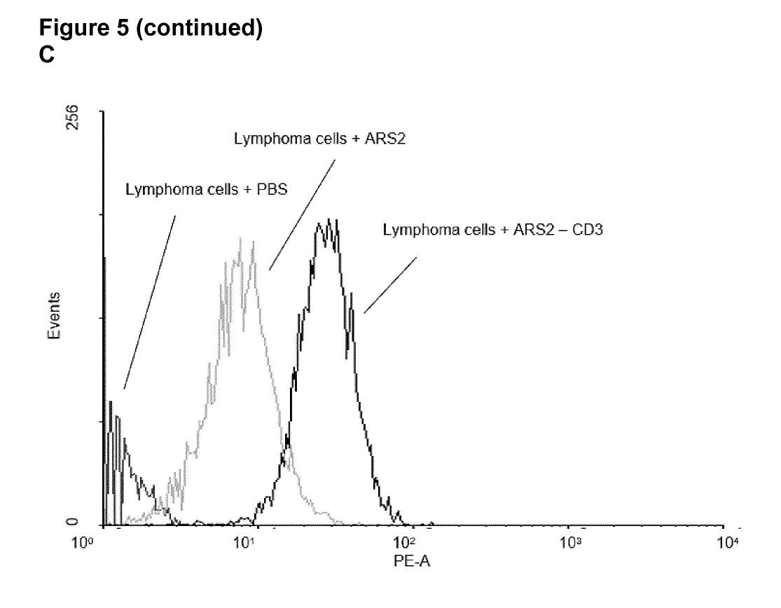

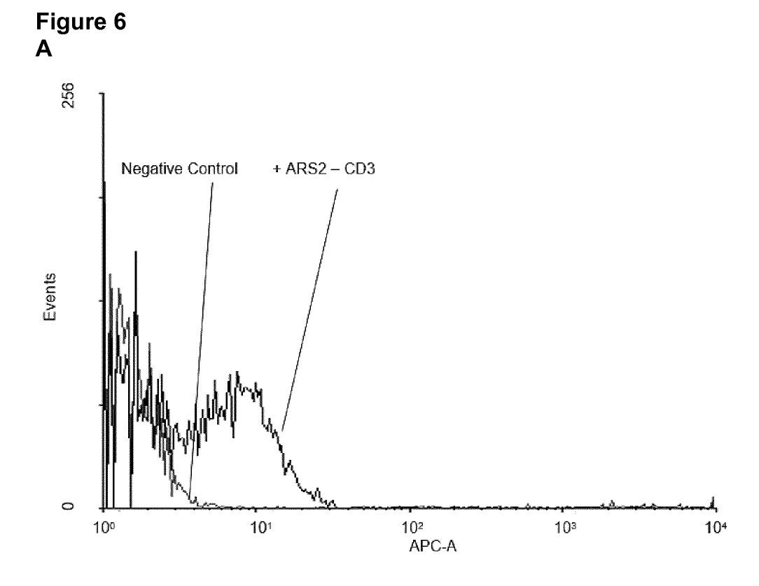

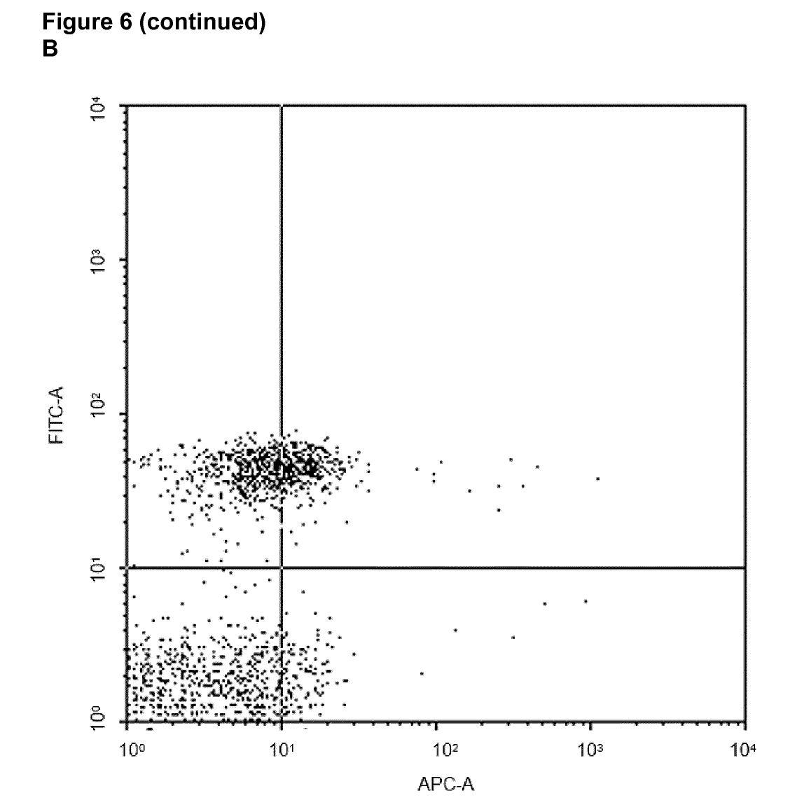

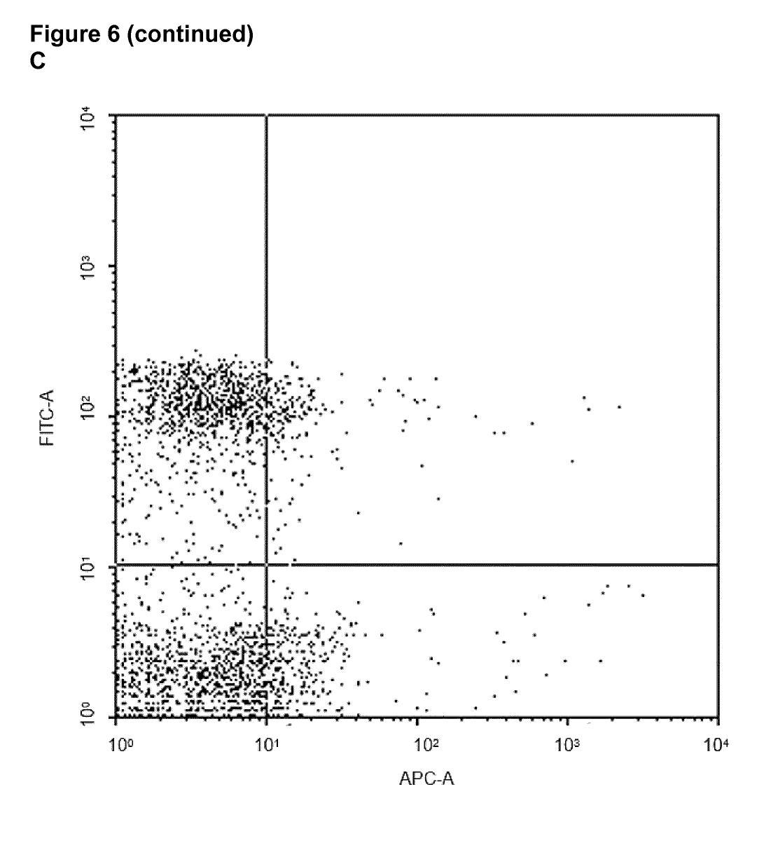

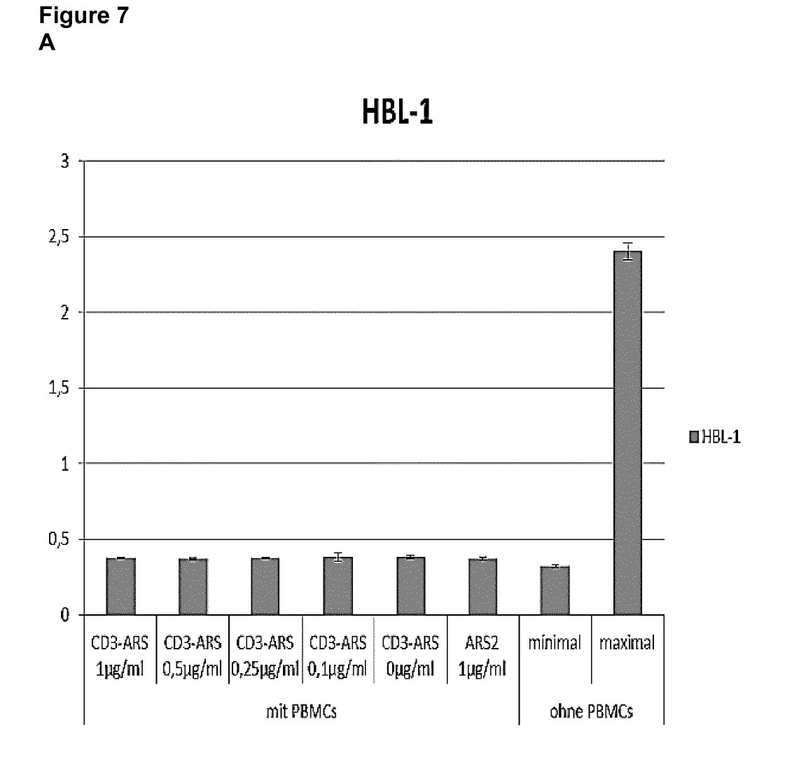

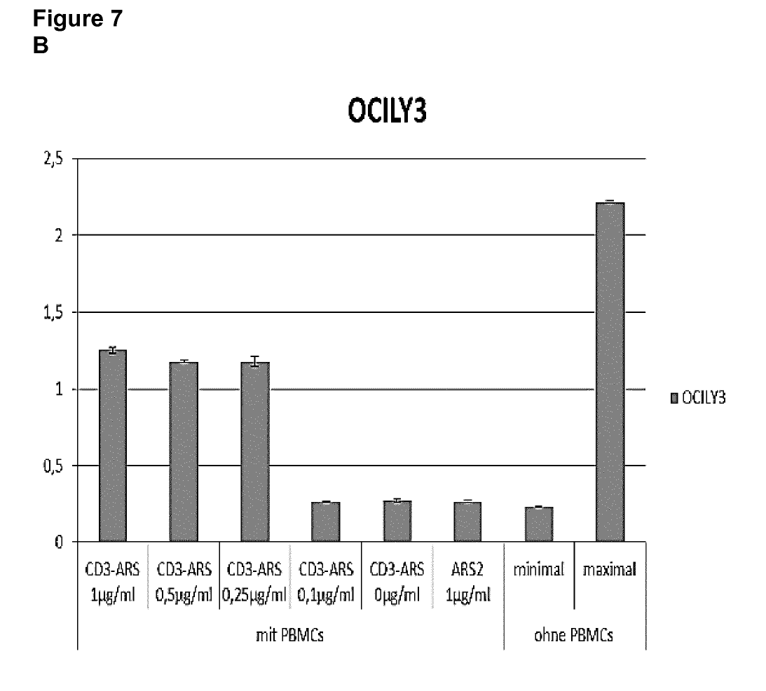

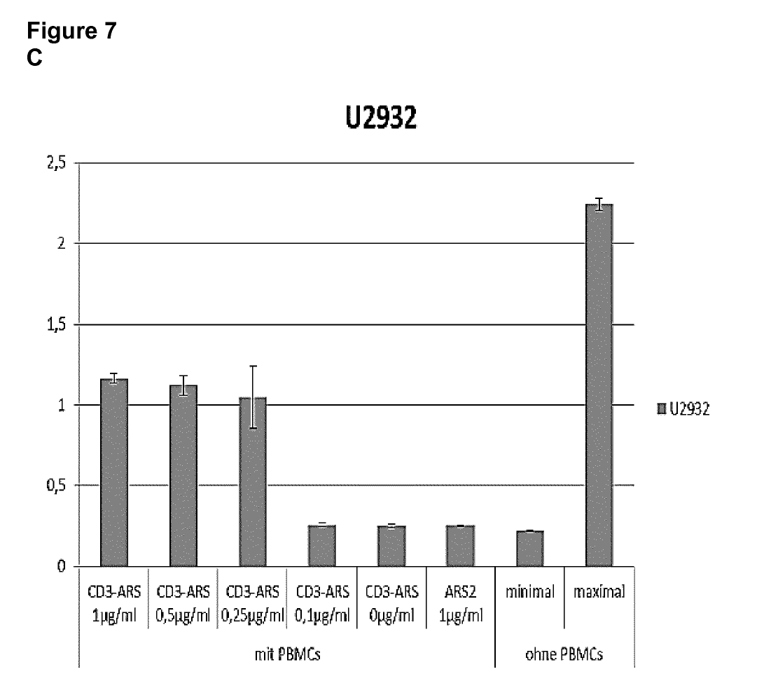

Our CD3—ARS2 bispecific construct binds simultaneously CD3 on T cells and ARS2 reactive lymphoma cell lines via their B cell receptor. It induces rapid cytotoxicity exclusively in ARS2 reactive lymphoma cell lines in concentrations as low as 250 ng/ml with an effector—target ratio of 10:1 (FIG. 5, 6). Specific cell mediated cytotoxicity reached 40% after 4 hours. Lymphoma cell lines with BCRs of a different specificity are not affected (FIG. 7).

1.-25. (canceled)

26. A conjugate comprising an ARS2 antigen or fragment thereof conjugated to a therapeutic agent for use in the treatment of diffuse large B cell lymphoma.

27. The conjugate of claim 26, wherein the ARS2 antigen is posttranslationally modified or unmodified.

28. The conjugate of claim 26, wherein the ARS2 antigen is a fragment derived from ARS2 protein.

29. A conjugate comprising a LRPAP1 antigen or fragment thereof conjugated to a therapeutic agent for use in the treatment of Mantle cell lymphoma.

30. The conjugate of claim 29, wherein the LRPAP1 antigen is posttranslationally modified or unmodified.

31. The conjugate of claim 29, wherein the LRPAP1 antigen is a fragment derived from LRPAP1 protein.

32. A B-cell receptor antigen conjugate comprising a B-cell receptor antigen conjugated to a therapeutic agent for use in treatment of a B-cell malignancy in a patient in need thereof, wherein
the B-cell receptor antigen conjugate comprises an ARS2 antigen or fragment thereof for use in the treatment of diffuse large B cell lymphoma, or
the B-cell receptor antigen conjugate comprises a LRPAP1 antigen or fragment thereof for use in the treatment of Mantle cell lymphoma, or
the B-cell receptor antigen conjugate comprises an antigen selected from the group consisting of MARK3, NCOR2, CACYBP, FAM32A, MYH2a, GLDC, PC9, LLP, Notch2, SMCHD1, MAZ, ACTINgamma, MTUS1, PHF20, PRKCSH, and fragment thereof for use in the treatment of chronic lymphocytic leukemia, or
the B-cell receptor antigen conjugate comprises an antigen selected from the group consisting of SAMD14, Neurabin1, GCN1L1 and fragment thereof for use in the treatment of Central Nervous System lymphomas, or
the B-cell receptor antigen conjugate comprises an antigen selected from the group consisting of PC3, RPS17a, RPOC-Moraxella and fragment thereof for use in the treatment of nodular lymphocyte predominant Hodgkin's lymphoma, or
the B-cell receptor antigen conjugate comprises an OPTN antigen for use in the treatment of follicular lymphomas.

33. The conjugate of claim 32, wherein the therapeutic agent is one of a radionuclide, a binding agent, a CAR T cell, a toxin, or a cytotoxic agent.

34. The conjugate of claim 33, wherein the binding agent is capable of binding to an immunologic effector cell.

35. The conjugate of claim 33, wherein the binding agent is one of a CD3 binding agent, a CD16 binding agent or a CD38 binding agent.

36. The conjugate of claim 33, wherein the binding agent is one of a monoclonal antibody, a polyclonal antibody, a single chain antibody, a ScFv, a minibody, a Fv, a Fab, a Fab′, or a F(ab′)2 fragment.

37. The conjugate of claim 33, wherein the toxin or the cytotoxic agent is one of an enidyene, duocarmycin, methothrexate, doxorubicin, melphalan, chlorambucil, ARA-C, vindesine, mitomycin C, cisplatin, etoposide, bleomycin, vedotin, emtansin, 5-fluorouracil, or a tyrosine kinase inhibitor.

38. The conjugate of claim 33, wherein the therapeutic agent is capable of reducing the number of malignant B-cells, and/or inhibiting activation or proliferation of malignant B-cells, and/or killing malignant B-cells, in a patient.

39. A pharmaceutical composition comprising the conjugate of claim 32 and a pharmaceutically acceptable excipient.

40. A method of treating diffuse large B cell lymphoma, the method comprising administering a therapeutically effective amount of the conjugate of claim 26.

41. A method of treating Mantle cell lymphoma, the method comprising administering a therapeutically effective amount of the conjugate of claim 29.

42. A method of treating a B-cell malignancy, the method comprising administering a therapeutically effective amount of the B-cell receptor antigen conjugate of claim 32.

43. The method of claim 42, wherein administration results in reduction of the number of malignant B-cells, and/or inhibition of activation or proliferation of malignant B-cells, and/or killing malignant B-cells.

44. A non-invasive method for identifying a patient suffering from a B-cell malignancy, being disposed to respond favorably to a conjugated B-cell receptor antigen, the method comprising
a) providing a labeled B-cell receptor antigen conjugate, wherein the B-cell receptor antigen conjugate is the conjugate of claim 32;
b) adding the labeled B-cell receptor antigen conjugate to a sample of a patient comprising malignant B-cells; and
c) assessing binding, internalization or binding and internalization of the labeled B-cell receptor antigen conjugate to the malignant B-cells,
wherein patients whose malignant B-cells bind to and/or internalize the labeled B-cell receptor antigen conjugate are disposed to respond favorably to a treatment with the conjugated B-cell receptor antigen, whereas patients whose malignant B-cells do not bind and/or internalize the labeled BCR antigen conjugate are not disposed to respond favorably to a treatment with the conjugated B-cell receptor antigen.


SEQUENCE LISTING (6 sequences)

SEQ ID NO: 1 - DNA, 33 bp, Artificial Sequence
  Primer
aagcttgccaccatgcaggtccagctgcagcag

SEQ ID NO: 2 - DNA, 78 bp, Artificial Sequence
  Primer
ggatccaccaccaccggagccgccgccgccagaaccaccaccaccagaaccaccaccacctgttgttttggctgagga

SEQ ID NO: 3 - DNA, 26 bp, Artificial Sequence
  Primer
ggatcccaaattgttctcacccagtc

SEQ ID NO: 4 - DNA, 69 bp, Artificial Sequence
  Primer
gaattcgatccgccaccgccagagccacctccgcctgaaccgcctccaccagttggtgcagtatcagcc

SEQ ID NO: 5 - DNA, 30 bp, Artificial Sequence
  Primer
gatatcatgaaggaagccaaaaagagtagc

SEQ ID NO: 6 - DNA, 52 bp, Artificial Sequence
  Primer
cccgggttaatggtggtggtgatgatgagcgctctctgactccgactcagac